(19) United States
(12) Patent Application Publication
(10) Pub. No.: US 2021/0310883 A1
(43) Pub. Date: Oct. 7, 2021

(54) STRAIN SENSOR

(71) Applicant: Murata Manufacturing Co., Ltd., Kyoto (JP)

(72) Inventors: Takayoshi OBATA, Kyoto (JP); Toru SHIMUTA, Kyoto (JP)

(21) Appl. No.: 17/354,048

(22) Filed: Jun. 22, 2021

Related U.S. Application Data
(63) Continuation of application No. PCT/JP2019/038235, filed on Sep. 27, 2019.

(30) Foreign Application Priority Data
Feb. 12, 2019 (JP) 2019-022435

(51) Int. Cl. G01L 1/22

(52) U.S. Cl. CPC G01L 1/22; A61B 5/11

(57) ABSTRACT

The present disclosure relates to a strain sensor. The strain sensor includes a sensor sheet provided with a sensing portion including a detection portion that expands and contracts in a predetermined direction according to a strain of an object to be measured and that detects a strain in the expansion and contraction direction, and a fixing member having a first main surface and a second main surface opposite to the first main surface. The sensor sheet is fixed so as to at least partially overlap the first main surface of the fixing member. A tensile load of the fixing member is greater than a tensile load of the sensing portion of the sensor sheet.

### CROSS REFERENCE TO RELATED APPLICATION

This is a continuation of International Application No. PCT/JP2019/038235 filed on Sep. 27, 2019 which claims priority from Japanese Patent Application No. 2019-022435 filed on Feb. 12, 2019. The contents of these applications are incorporated herein by reference in their entireties.

### BACKGROUND OF THE DISCLOSURE

### Field of the Disclosure

The present disclosure relates to a strain sensor.

### Description of the Related Art

In recent years, strain sensors are used in detection, control, and the like of the motions of bodies and the motions of robots. For example, Patent Document 1 describes a stretchable circuit board that is used in a state where a stretchable substrate is attached to a living body.

Patent Document 1: Japanese Unexamined Patent Application Publication No. 2016-145725

### BRIEF SUMMARY OF THE DISCLOSURE

As for the strain sensor as described in Patent Document 1, when an elastic substance is interposed between the sensor and an object of which the motion is measured, the motion is absorbed by the interposed substance, with the result that the followability of the sensor can be impaired. In this case, the motion of the object cannot be accurately detected. When, for example, the motion of a joint or cartilage of a human body is measured, the motion is detected by the sensor via a skin on the surface of the joint or cartilage. In this case, the followability of the sensor depends on individual differences in the elasticity, shape, such as wrinkles, and the like of the skin, with the result that different detection results can be obtained.

It is an object of the present disclosure to provide a strain sensor with less variations in detection result even when an elastic substance is interposed between the strain sensor and a measuring object as described above.

The present disclosure includes the following aspects.

[1] A strain sensor includes a sensor sheet provided with a sensing portion including a detection portion that expands and contracts in a predetermined direction according to a strain of an object to be measured and that detects a strain in the expansion and contraction direction, and a fixing member having a first main surface and a second main surface opposite to the first main surface. The sensor sheet is fixed so as to at least partially overlap the first main surface of the fixing member. A tensile load of the fixing member is greater than a tensile load of the sensing portion of the sensor sheet.

[2] In the strain sensor according to the above-described [1], the tensile load of the sensing portion is less than a tensile load of the object to be measured.

[3] In the strain sensor according to the above-described [1] or [2], a tensile load of the strain sensor in a region in which the sensing portion is present is less than or equal to 0.10 N/mm at a strain of 5%, less than or equal to 0.15 N/mm at a strain of 10%, and less than or equal to 0.25 N/mm at a strain of 20% along an expansion and contraction direction of the detection portion, and a compressive load of the fixing member is greater than or equal to 0.005 N/mm at a strain of 5%, greater than or equal to 0.01 N/mm at a strain of 10%, and greater than or equal to 0.03 N/mm at a strain of 20% along the expansion and contraction direction of the detection portion.

[4] A strain sensor includes a sensor sheet provided with a sensing portion including a detection portion that expands and contracts in a predetermined direction according to a strain of an object to be measured and that detects a strain in the expansion and contraction direction, and a non-sensing portion that is located on each end of the sensing portion and that supports the sensing portion. The sensing portion is easier to deform than the non-sensing portion.

[5] In the strain sensor according to the above-described [4], where a Young's modulus of the sensing portion is Y1, a thickness of the sensing portion is T1, a Young's modulus of the non-sensing portion is Y2, and a thickness of the non-sensing portion is T2, a product F1 of Y1 and T1 is less than a product F2 of Y2 and T2.

[6] The strain sensor according to the above-described [4] or [5] further includes a fixing member having a first main surface and a second main surface opposite to the first main surface. The sensor sheet is fixed so as to at least partially overlap the first main surface of the fixing member. In plan view, a portion at which the sensing portion and the fixing member overlap is easier to deform than a portion at which the non-sensing portion and the fixing member overlap.

[7] In the strain sensor according to any one of the above-described [1] to [3], and [6], the fixing member is a sponge material.

[8] In the strain sensor according to the above-described [7], a thickness of the fixing member is greater than or equal to 1 mm and less than or equal to 5 mm.

[9] In the strain sensor according to any one of the above-described [1] to [3], and [8], an outer shape of the fixing member and an outer shape of the sensor sheet overlap in plan view.

[10] In the strain sensor according to any one of the above-described [1] to [3], and [9], the fixing member is present so as to at least overlap the entire sensor sheet in plan view.

[11] In the strain sensor according to any one of the above-described [1] to [3], and [10], the fixing member is present so as to surround the sensing portion of the sensor sheet in plan view.

[12] In the strain sensor according to any one of the above-described [1] to [3], and [11], a plurality of the detection portions is present.

[13] In the strain sensor according to the above-described [12], the plurality of detection portions is disposed parallel to each other.

[14] In the strain sensor according to any one of the above-described [1] to [12], the sensing portion includes a plurality of the detection portions, and at least one of the detection portions and another one of the detection portions expand and contract in different directions.

[15] In the strain sensor according to the above-described [14], at least one or some of the plurality of detection portions are disposed parallel to each other, and another one or some of the detection portions are disposed so as to intersect with a region extending in a length direction from all the detection portions disposed parallel to each other.

[16] In the strain sensor according to the above-described [12], the plurality of detection portions is disposed such that expansion and contraction directions of the detection portions are radial.

[17] In the strain sensor according to the above-described [1] to [16], the detection portion is a detection conductor of which a resistance value changes according to expansion and contraction of the detection portion.

[18] In the strain sensor according to any one of the above-described [1] to [17], the sensing portion is placed in a state where a tensile stress is applied along an expansion and contraction direction of the detection portion.

[19] In the strain sensor according to any one of the above-described [1] to [18], the sensing portion has a plurality of slits provided in a direction that intersects with an expansion and contraction direction of the detection portion.

[20] In the strain sensor according to any one of the above-described [1] to [3], and [6] to [19], a hysteresis of an elastic modulus of the fixing member during expansion and contraction of the fixing member is smaller than a hysteresis of an elastic modulus of the sensing portion during expansion and contraction of the sensing portion.

According to the present disclosure, a strain sensor with less variations in detection result is provided even when an elastic substance is interposed between a strain sensor and a measuring object.

### BRIEF DESCRIPTION OF THE SEVERAL VIEWS OF THE DRAWINGS

FIG. 1 is a plan view showing the configuration of a strain sensor of a first embodiment according to the present disclosure.

FIG. 2 is a plan view showing the configuration of a sensor unit in the strain sensor of the first embodiment according to the present disclosure.

FIG. 3 is a plan view showing a fixing member in the strain sensor of the first embodiment according to the present disclosure.

FIG. 4 is a plan view showing the configuration of a strain sensor of a second embodiment according to the present disclosure.

FIG. 5 is a plan view showing a fixing member in the strain sensor of the second embodiment according to the present disclosure.

FIG. 6 is a plan view showing the configuration of a strain sensor of a third embodiment according to the present disclosure.

FIG. 7 is a plan view showing the configuration of a strain sensor of a fourth embodiment according to the present disclosure.

FIG. 8 is a plan view showing the configuration of a strain sensor of a fifth embodiment according to the present disclosure.

FIG. 9 is a plan view showing the configuration of a strain sensor of a sixth embodiment according to the present disclosure.

FIG. 10 is a plan view showing the configuration of a strain sensor of a seventh embodiment according to the present disclosure.

FIG. 11 is a plan view showing the configuration of a strain sensor of an eighth embodiment according to the present disclosure.

FIG. 12 is a plan view showing the configuration of a strain sensor of a ninth embodiment according to the present disclosure.

FIG. 13 is a plan view showing the configuration of a strain sensor of a tenth embodiment according to the present disclosure when viewed from the back side of the strain sensor.

FIG. 14 is a plan view showing the configuration of a strain sensor of an eleventh embodiment according to the present disclosure.

FIG. 15 is a view showing a usage state when a strain sensor according to the present disclosure is used as a swallowing sensor.

FIG. 16 is a graph showing measurement results of the motion of a throat of subject A with a sensor sheet.

FIG. 17 is a graph showing measurement results of the motion of a throat of subject B with a sensor sheet.

FIG. 18 is a graph showing measurement results of the motion of a throat when the strain sensor according to the present disclosure is attached with no creases.

FIG. 19 is a graph showing measurement results of the motion of a throat when the strain sensor according to the present disclosure is attached with intentionally created creases.

FIG. 20 is a graph showing measurement results of the motion of a throat when water is swallowed while the strain sensor according to the present disclosure is attached.

### DETAILED DESCRIPTION OF THE DISCLOSURE

A strain sensor according to the present disclosure is a sensor that is attached to an object to be measured and that detects the motion of a measurement region of the object to be measured due to the motion of a measuring object.

The “object to be measured” means an object to which the strain sensor according to the present disclosure is directly attached. The “measuring object” means an object to detect motion with the strain sensor. When, for example, the motion of a joint or cartilage of a human body is measured, the strain sensor according to the present disclosure is attached to the body surface of a point where the joint or cartilage of the human body is present, a measuring object is the joint or the cartilage, and an object to be measured is the human body. An object to be measured and a measuring object can be the same. The “measurement region” means a measurement target region of an object to be measured, and a sensing portion of the strain sensor of the present disclosure is in contact with the measurement region.

The strain sensor according to the present disclosure includes a sensor sheet provided with the sensing portion including a detection portion that expands and contracts in a predetermined direction according to a strain of an object to be measured and that detects a strain in the expansion and contraction direction, and a fixing member having a first main surface and a second main surface opposite to the first main surface. In the strain sensor according to the present disclosure, the sensor sheet is fixed so as to at least partially overlap the first main surface of the fixing member, and a tensile load of the fixing member is greater than a tensile load of the sensing portion of the sensor sheet.

The fixing member of the strain sensor according to the present disclosure has a greater tensile load than the sensor sheet. In other words, the fixing member is more difficult to expand than the sensor sheet. As described above, when an elastic substance is interposed between an existing strain sensor and a measuring object, the followability of the sensor is impaired, with the result that the motion of the measuring object may not be accurately detected. On the other hand, the strain sensor according to the present disclosure measures a strain of an object to be measured via the fixing member lower in stretchability than the sensor sheet. Thus, the strain sensor according to the present disclosure equalizes a degree to which the motion of a measuring object is absorbed by an elastic intervening substance, with the result that the strain sensor is capable of measuring a strain with reduced variations.

The sensor sheet is a remarkably thin stretchable material with a thickness of several tens of micrometers and has a low mechanical strength. In addition, to enhance the followability, the sensor sheet may be enhanced in elasticity by, for example, slitting. In this case, the mechanical strength further decreases. When the mechanical strength is low, there is a problem that the sensor sheet easily breaks at the time of handling, particularly, at the time of reattaching. With the strain sensor according to the present disclosure, the sensor sheet having a low mechanical strength is attached to the fixing member having a high mechanical strength, so a load on the sensor sheet at the time of handling the strain sensor is reduced. In addition, when the maximum elongation of the fixing member is made less than the maximum elongation of the sensor sheet, a strain of the sensor sheet does not reach a breaking strain even when an excessive load is applied.

Hereinafter, the strain sensor according to the present disclosure will be described in detail with reference to the drawings. However, the shapes, arrangement, and the like of a strain sensor and component elements of each of the embodiments are not limited to the illustrated examples.

### First Embodiment

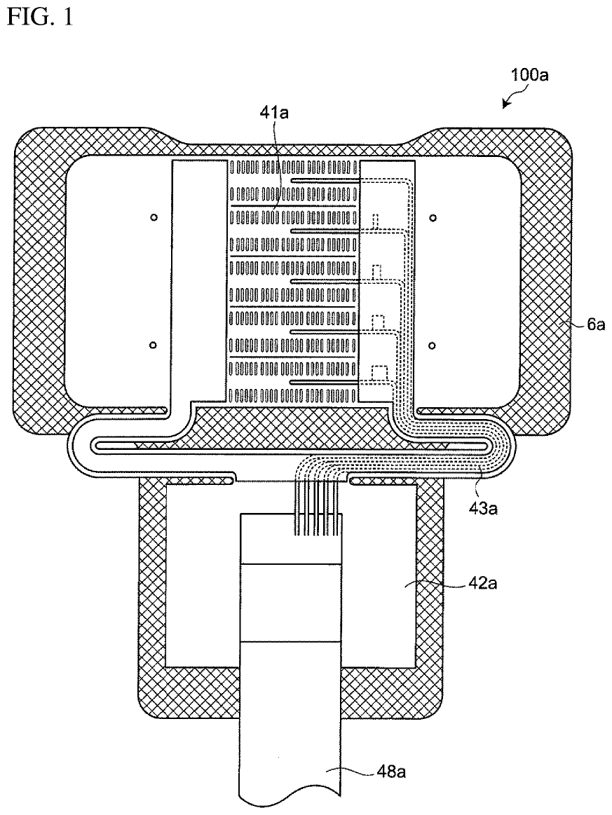

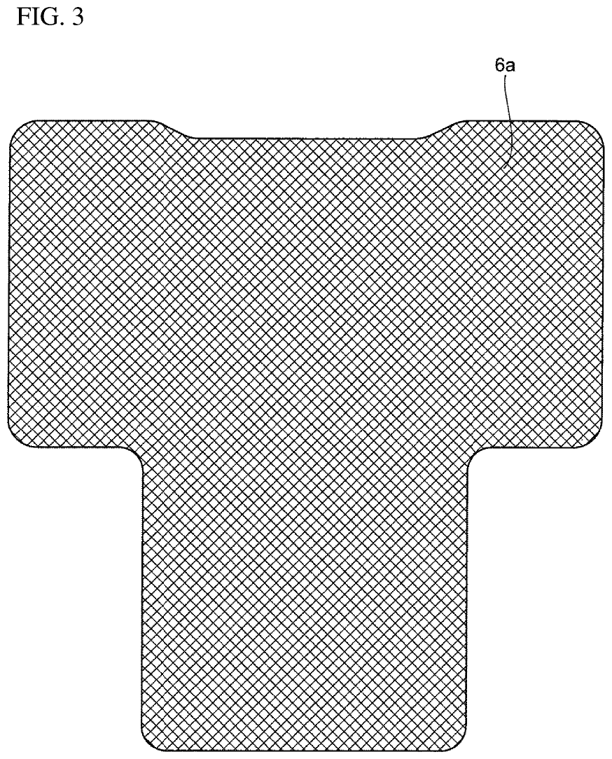

As shown in FIG. 1 to FIG. 3, a strain sensor 100a of the first embodiment is a strain sensor provided with a sensor unit 4a and a fixing member 6a.

The sensor unit 4a includes a sensor sheet 41a, a terminal portion 42a, and a connection portion 43a. The sensor sheet 41a includes a sensing portion 45a that detects a strain in a predetermined direction, and non-sensing portions 46a, 47a respectively located on both ends of the sensing portion 45a. The sensor sheet 41a is coupled to the terminal portion 42a via the connection portion 43a.

The fixing member 6a has a first main surface and a second main surface opposite to the first main surface. The tensile load of the fixing member 6a is greater than the tensile load of the sensing portion 45a of the sensor sheet 41a.

The sensor unit 4a is fixed to the first main surface of the fixing member 6a by attaching the sensor sheet 41a and the terminal portion 42a to the fixing member 6a. The sensor sheet 41a is fixed such that the entire sensor sheet 41a overlaps the first main surface of the fixing member 6a. In other words, in plan view, the fixing member 6a is present so as to overlap the entire sensor sheet 41a. The plan view means a view of the strain sensor in a direction perpendicular to the main surface of the fixing member.

The strain sensor 100a of the first embodiment is used in a state where the second main surface of the fixing member 6a is attached to an object to be measured such that the sensing portion 45a of the sensor sheet 41a is located in a measurement region of the object to be measured.

Hereinafter, specific configurations of the sensor unit 4a, the fixing member 6a, and the strain sensor 100a will be described.

(Sensor Unit)

As described above, the sensor unit 4a includes the sensor sheet 41a, the terminal portion 42a, and the connection portion 43a.

The sensor sheet 41a includes a substrate 51a having a first main surface and a second main surface opposite to the first main surface, and conductors 52a provided on the first main surface of the substrate 51a.

The constituent material of the substrate 51a is preferably a stretchable material having a low elastic modulus and preferably contains, for example, a stretchable material having a low elastic modulus, such as polyurethane, acrylic, and silicone resin.

The thickness of the substrate 51a is not limited and can be preferably greater than or equal to 10 μm and less than or equal to 200 μm, more preferably greater than or equal to 20 μm and less than or equal to 100 μm, and further preferably greater than or equal to 30 μm and less than or equal to 50 μm.

The conductors 52a extend to the connection portion 43a and the terminal portion 42a. In other words, each of the conductors 52a includes a terminal conductor 52a4 provided in the terminal portion 42a, a wiring conductor 52a3 provided in the connection portion 43a, a fixed conductor 52a2 provided in the non-sensing portion 46a, and a detection conductor 52a1 provided in the sensing portion 45a. More specifically, the conductors 52a extend from the terminal portion 42a to the sensing portion 45a of the sensor sheet via the connection portion 43a and the non-sensing portion 46a of the sensor sheet. In the sensing portion 45a, the conductors 52a extend from the right end toward the left, and are folded back near the center of the sensing portion 45a, and then return to the right end. Here, the right side in the drawing is defined as the right side of the sensing portion 45a. The conductors 52a returned to the right end extend to the terminal portion 42a via the non-sensing portion 46a of the sensor sheet and the connection portion 43a. The folded back conductors 52a are disposed parallel to one another. The detection conductors 52a1 expand and contract in a right and left direction of the sensing portion 45a according to expansion and contraction of the sensing portion 45a in the right and left direction. As the length of each detection conductor 52a1 changes, the resistance value changes. By detecting the change in the resistance value of each detection conductor 52a1, the amount of expansion and contraction of the sensing portion 45a, that is, a strain of an object to be measured, is detected. In other words, the detection conductors 52a1 make up a detection portion.

The constituent material of the detection conductors 52a1 in the conductors 52a is preferably a material having a large change in resistance value for expansion and contraction and is preferably, for example, a mixture containing metal particles, such as silver (Ag) and copper (Cu), and an elastomeric resin, such as silicone. When the detection conductors 52a1 are made of a mixture of metal particles and resin, not only a variation in contact points between the metal particles but also distances between the metal particles increase due to expansion and contraction of the sensing portion 45a, so the rate of increase or the rate of reduction in resistance value to a displacement is increased. In addition, when the detection conductors 52a1 are made of a mixture of metal particles and resin, a break due to a deformation is prevented by the stretchability of the resin.

The constituent material of a portion other than the detection conductors 52a1 in the conductors 52a, specifically, the fixed conductors 52a2, the wiring conductors 52a3, and the terminal conductors 52a4, may be the same as the constituent material of the detection conductors 52a1 or may be different from the constituent material of the detection conductors 52a1. When the conductors 52a except the detection conductors 52a1 are made of the same material as the detection conductors 52a1, the detection conductors 52a1 and the conductors 52a except the detection conductors 52a1 can be simultaneously formed in one step, so the manufacturing cost is reduced. When the conductors 52a except the detection conductors 52a1 are made of a constituent material different from the detection conductors 52a1, the conductors 52a can be configured to increase a variation in resistance value to a displacement of each of the detection conductors 52a1 and to prevent a break due to expansion and contraction, and the conductors 52a except the detection conductors 52a1 can be made of a material having a low resistance, so the detection of a highly accurate strain is possible.

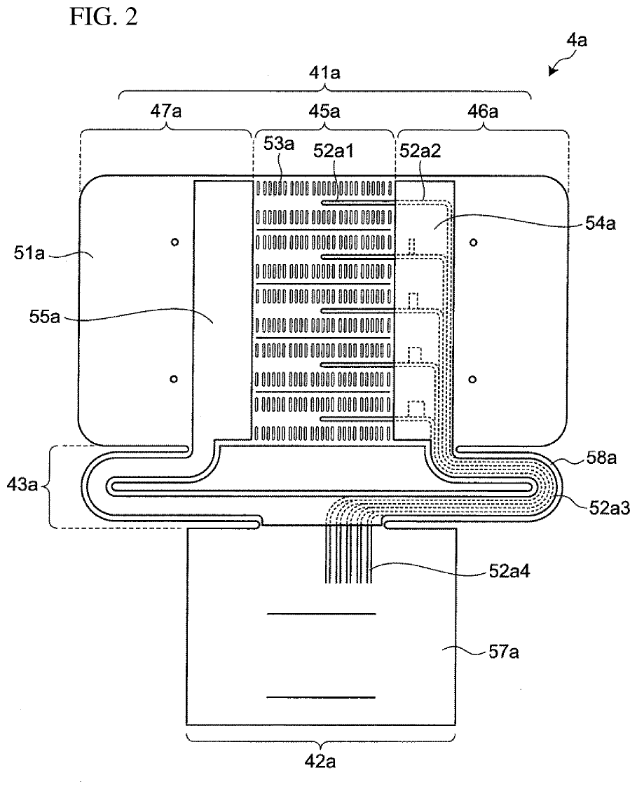

In the strain sensor 100a of the first embodiment, the number of conductors 52a is five. In other words, the strain sensor 100a includes a plurality of detection portions. The detection portions are disposed parallel to one another at equal intervals in a vertical direction in the sensing portion 45a. The vertical direction means an up and down direction in FIG. 1 and FIG. 2. By providing the plurality of detection portions, a strain over a wider range can be detected, or, when detection in a range of the same area is performed, accuracy can be further improved.

The sensing portion 45a is a region to measure a change in the shape of an object to be measured. The outer shape of the sensing portion 45a is set in consideration of the range of a measurement region. The followability of the sensing portion 45a is set in consideration of the elasticity of the object to be measured.

The sensing portion 45a has a plurality of slits 53a provided in a direction that intersects with the expansion and contraction direction of the detection portions. The sensing portion 45a has a shape and a structure easier to deform than the surroundings by providing the sensing portion 45a with the slits 53a, so the followability of the sensing portion 45a is enhanced.

In the strain sensor 100a of the first embodiment, as shown in FIG. 1, the sensing portion 45a includes the detection portions made up of the detection conductors 52a1, and a low-elastic modulus portion configured not to restrain a deformation of the detection portions to a strain and not to restrain a deformation of an object to be measured. Here, in the specification, the “low-elastic modulus” in the case of referring to low-elastic modulus or imparting a low-elastic modulus property in the low-elastic modulus portion means that the elastic modulus is lower than the elastic modulus of the non-sensing portions 46a, 47a.

The non-sensing portions 46a, 47a support the sensing portion 45a such that, when a measurement region of an object to be measured expands and contracts, the sensing portion 45a expands and contracts according to the expansion and contraction. In the strain sensor 100a of the first embodiment, the non-sensing portions 46a, 47a are provided respectively on both sides of the sensing portion 45a in the expansion and contraction direction of the detection conductors 52a1 (that is, the detection portions). The non-sensing portions 46a, 47a respectively include restraint portions 54a, 55a such that, when a measurement region in an object to be measured expands and contracts, a strain according to the expansion and contraction in the measurement region is detected under no influence of expansion and contraction in a region other than the measurement region. As shown in FIG. 2, the restraint portions 54a, 55a are respectively provided in the non-sensing portions 46a, 47a. The restraint portions 54a, 55a are preferably provided in proximity to the sensing portion 45a. Thus, an influence of a portion other than a measurement region in an object to be measured is reduced, with the result that a strain in the measurement region can be accurately measured.

The terminal portion 42a includes the substrate 57a and the terminal conductors 52a4. The terminal conductors 52a4 are provided on one of the main surfaces of the substrate 57a.

The constituent material of the substrate 57a is not limited and can be the same material as the constituent material of the substrate 51a, for example, polyurethane, acrylic, silicone resin, or the like.

The connection portion 43a includes the substrate 58a and the wiring conductors 52a3. The wiring conductors 52a3 are provided on one of the main surfaces of the substrate 58a. The connection portion 43a is provided to couple the sensor sheet 41a to the terminal portion 42a and to electrically connect the detection conductors 52a1 in the sensor sheet 41a and the terminal conductors 52a4 in the terminal portion 42a.

(Fixing Member)

The fixing member 6a is a sheet-shaped member having a first main surface and a second main surface opposite to the first main surface.

The tensile load of the fixing member 6a is greater than the tensile load of the sensor sheet 41a. In other words, the fixing member 6a is more difficult to expand than the sensor sheet 41a. With such a configuration, the degree to which the motion is absorbed by an elastic intervening substance is equalized, so variations in results of strain measurement are reduced. When, for example, the measuring object is a joint or a cartilage, the motion is detected by the sensor via a skin on the surface of the joint or the cartilage. Depending on individual differences in the elasticity, shape, such as winkles, and the like, of the skin, the followability of the sensor can vary and different measurement results can be obtained. Even when there are individual differences in this way, the strain sensor according to the present disclosure suppresses variations in measurement results.

The constituent material of the fixing member 6a may be rubber, sponge, or the like.

Urethane rubber or silicon rubber may be the rubber.

The sponge may be nitrile rubber sponge (NBR sponge), chloroprene rubber sponge (CR sponge), ethylene rubber sponge (EPDM rubber sponge), or the like and is preferably chloroprene rubber sponge.

The sponge may be any one of a type of closed-cell foam and a type of open-cell foam.

The fixing member 6a preferably has an Asker C hardness of less than or equal to 30 and more preferably has an Asker C hardness of less than or equal to 25. By reducing the Asker C hardness of the fixing member, the sponge is softer, so the restraint on the deformation of an object to be measured is suppressed. The fixing member preferably has an Asker C hardness of greater than or equal to 10 and more preferably has an Asker C hardness of greater than or equal to 20. By increasing the Asker C hardness of the fixing member, the degree to which the motion of an object to be measured is absorbed by an elastic intervening substance is equalized, so variations in results of strain measurement are reduced.

The Asker C hardness can be measured in conformity with JIS K 7312.

The fixing member 6a, at a strain of 5%, preferably has a tensile load of less than or equal to 0.10 N/mm, more preferably has a tensile load of less than or equal to 0.08 N/mm, and further preferably has a tensile load of less than or equal to 0.06 N/mm. By setting the tensile load of the fixing member in the above-described range, the followability of the strain sensor to the motion of an object to be measured improves, so further highly accurate detection is possible.

The fixing member 6a, at a strain of 5%, preferably has a tensile load of greater than or equal to 0.01 N/mm, more preferably has a tensile load of greater than or equal to 0.02 N/mm, and further preferably has a tensile load of greater than or equal to 0.03 N/mm. By increasing the tensile load of the fixing member, the degree to which the motion of an object to be measured is absorbed by an elastic intervening substance is equalized, so variations in results of strain measurement are reduced.

The fixing member 6a, at a strain of 10%, preferably has a tensile load of less than or equal to 0.15 N/mm, more preferably has a tensile load of less than or equal to 0.12 N/mm, and further preferably has a tensile load of less than or equal to 0.08 N/mm. By setting the tensile load of the fixing member in the above-described range, the followability of the strain sensor to the motion of an object to be measured improves, so further highly accurate detection is possible.

The fixing member 6a, at a strain of 10%, preferably has a tensile load of greater than or equal to 0.01 N/mm, more preferably has a tensile load of greater than or equal to 0.03 N/mm, and further preferably has a tensile load of greater than or equal to 0.05 N/mm. By increasing the tensile load of the fixing member, the degree to which the motion of an object to be measured is absorbed by an elastic intervening substance is equalized, so variations in results of strain measurement are reduced.

The fixing member 6a, at a strain of 20%, preferably has a tensile load of less than or equal to 0.25 N/mm, more preferably has a tensile load of less than or equal to 0.20 N/mm, and further preferably has a tensile load of less than or equal to 0.15 N/mm. By setting the tensile load of the fixing member in the above-described range, the followability of the strain sensor to the motion of an object to be measured improves, so further highly accurate detection is possible.

The fixing member 6a, at a strain of 20%, preferably has a tensile load of greater than or equal to 0.01 N/mm, more preferably has a tensile load of greater than or equal to 0.05 N/mm, and further preferably has a tensile load of greater than or equal to 0.10 N/mm. By increasing the tensile load of the fixing member, the degree to which the motion of an object to be measured is absorbed by an elastic intervening substance is equalized, so variations in results of strain measurement are reduced.

The tensile load can be measured by Automatic horizontal servo stand JSH-H1000 made by Japan Instrumentation System Co., Ltd.

In a preferred mode, the tensile load of the fixing member 6a is greater than the tensile load of the sensor sheet 41a and less than the tensile load of an object to be measured, typically, the tensile load of the surface of an object to be measured.

The fixing member 6a, at a strain of 5%, preferably has a compressive load of greater than or equal to 0.005 N/mm, more preferably has a compressive load of greater than or equal to 0.01 N/mm, and further preferably has a compressive load of greater than or equal to 0.015 N/mm. By setting the compressive load of the fixing member in the above-described range, collapse of the fixing member by the stress of the sensor sheet is suppressed when the sensor sheet is fixed to the fixing member in a state where a tensile stress is applied.

The fixing member 6a, at a strain of 5%, preferably has a compressive load of less than or equal to 0.10 N/mm, more preferably has a compressive load of less than or equal to 0.08 N/mm, and further preferably has a compressive load of less than or equal to 0.06 N/mm. By setting the compressive load of the fixing member in the above-described range, the followability of the strain sensor to the motion of an object to be measured improves, so further highly accurate detection is possible.

The fixing member 6a, at a strain of 10%, preferably has a compressive load of greater than or equal to 0.01 N/mm, more preferably has a compressive load of greater than or equal to 0.02 N/mm, and further preferably has a compressive load of greater than or equal to 0.03 N/mm. By setting the compressive load of the fixing member in the above-described range, collapse of the fixing member by the stress of the sensor sheet is suppressed when the sensor sheet is fixed to the fixing member in a state where a tensile stress is applied.

The fixing member 6a, at a strain of 10%, preferably has a compressive load of less than or equal to 0.15 N/mm, more preferably has a compressive load of less than or equal to 0.12 N/mm, and further preferably has a compressive load of less than or equal to 0.08 N/mm. By setting the compressive load of the fixing member in the above-described range, the followability of the strain sensor to the motion of an object to be measured improves, so further highly accurate detection is possible.

The fixing member 6a, at a strain of 20%, preferably has a compressive load of greater than or equal to 0.03 N/mm, more preferably has a compressive load of greater than or equal to 0.04 N/mm, and further preferably has a compressive load of greater than or equal to 0.05 N/mm. By setting the compressive load of the fixing member in the above-described range, collapse of the fixing member by the stress of the sensor sheet is suppressed when the sensor sheet is fixed to the fixing member in a state where a tensile stress is applied.

The fixing member 6a, at a strain of 20%, preferably has a compressive load of less than or equal to 0.25 N/mm, more preferably has a compressive load of less than or equal to 0.20 N/mm, and further preferably has a compressive load of less than or equal to 0.15 N/mm. By setting the compressive load of the fixing member in the above-described range, the followability of the strain sensor to the motion of an object to be measured improves, so further highly accurate detection is possible.

The compressive load can be measured by, for example, measuring a compressive load at the time when the bottom surface of a 10 mm diameter cylindrical tool is pressed against a 10 mm thick sample in the thickness direction at a distance corresponding to a strain of a predetermined magnitude (for example, 2 mm in the case of a strain of 20%).

The thickness of the fixing member 6a is preferably greater than or equal to 0.1 mm and less than or equal to 5.0 mm and more preferably greater than or equal to 1.0 mm and less than or equal to 3.0 mm. Particularly, when the fixing member is a sponge, the thickness of the fixing member is preferably greater than or equal to 1.0 mm and less than or equal to 3.0 mm. As the thickness of the fixing member is reduced, a strain is more accurately detected. On the other hand, as the thickness of the fixing member is increased, the mechanical strength of the strain sensor is more enhanced.

The fixing member 6a preferably has a breaking strain of higher than or equal to 130% and more preferably has a breaking strain of higher than or equal to 160%. By setting the breaking strain of the fixing member in the above-described range, the risk of breakage decreases, so a relatively large motion of an object to be measured can also be handled.

The fixing member preferably has a breaking strain of lower than or equal to 250% and more preferably has a breaking strain of lower than or equal to 200%.

In the present embodiment, the size of the fixing member 6a is greater in plan view than the size of the sensor sheet 41a. When the size of the fixing member is greater than the size of the sensor sheet, the entire sensor sheet can be attached onto the fixing member, so a further stable strain detection is possible, and the mechanical strength increases.

(Strain Sensor)

The strain sensor 100a of the first embodiment includes the sensor unit 4a and the fixing member 6a. The sensor sheet 41a and the terminal portion 42a of the sensor unit 4a are fixed so as to overlap the fixing member 6a. A flat cable 48a is connected to the terminal portion 42a.

The strain sensor 100a, at a strain of 5%, preferably has a tensile load of less than or equal to 0.10 N/mm, more preferably has a tensile load of less than or equal to 0.08 N/mm, and further preferably has a tensile load of less than or equal to 0.065 N/mm along the expansion and contraction direction of the detection portions in the region in which the sensing portion 45a is present. By setting the tensile load of the fixing member to which the sensor sheet is fixed in the above-described range, the followability of the strain sensor to the motion of an object to be measured improves, so further highly accurate detection is possible. Here, the “fixing member 6a to which the sensor sheet 41a is fixed” means a portion of the fixing member, to which the sensor sheet is fixed. A detection direction means a direction in which the detection conductors expand (the right and left direction in FIG. 1).

The strain sensor 100a, at a strain of 5%, preferably has a tensile load of greater than or equal to 0.01 N/mm, more preferably has a tensile load of greater than or equal to 0.03 N/mm, and further preferably has a tensile load of greater than or equal to 0.05 N/mm along the expansion and contraction direction of the detection portions in the region in which the sensing portion 45a is present. By increasing the tensile load of the fixing member, the degree to which the motion of an object to be measured is absorbed by an elastic intervening substance is equalized, so variations in results of strain measurement are reduced.

The strain sensor 100a, at a strain of 10%, preferably has a tensile load of less than or equal to 0.15 N/mm, more preferably has a tensile load of less than or equal to 0.13 N/mm, and further preferably has a tensile load of less than or equal to 0.11 N/mm along the expansion and contraction direction of the detection portions in the region in which the sensing portion 45a is present. By setting the tensile load of the fixing member in the above-described range, the followability of the strain sensor to the motion of an object to be measured improves, so further highly accurate detection is possible.

The strain sensor 100a, at a strain of 10%, preferably has a tensile load of greater than or equal to 0.01 N/mm, more preferably has a tensile load of greater than or equal to 0.04 N/mm, and further preferably has a tensile load of greater than or equal to 0.07 N/mm along the expansion and contraction direction of the detection portions in the region in which the sensing portion 45a is present. By increasing the tensile load of the fixing member, the degree to which the motion of an object to be measured is absorbed by an elastic intervening substance is equalized, so variations in results of strain measurement are reduced.

The strain sensor 100a, at a strain of 20%, preferably has a tensile load of less than or equal to 0.25 N/mm, more preferably has a tensile load of less than or equal to 0.22 N/mm, and further preferably has a tensile load of less than or equal to 0.19 N/mm along the expansion and contraction direction of the detection portions in the region in which the sensing portion 45a is present. By setting the tensile load of the fixing member in the above-described range, the followability of the strain sensor to the motion of an object to be measured improves, so further highly accurate detection is possible.

The strain sensor 100a, at a strain of 20%, preferably has a tensile load of greater than or equal to 0.01 N/mm, more preferably has a tensile load of greater than or equal to 0.05 N/mm, and further preferably has a tensile load of greater than or equal to 0.10 N/mm along the expansion and contraction direction of the detection portions in the region in which the sensing portion 45a is present. By increasing the tensile load of the fixing member, the degree to which the motion of an object to be measured is absorbed by an elastic intervening substance is equalized, so variations in results of strain measurement are reduced.

In a preferred mode, the tensile load in the sensing portion 45a of the strain sensor 100a is less than the tensile load of an object to be measured, typically, the tensile load of the surface of an object to be measured.

In a preferred mode, the sensor sheet 41a is attached to the fixing member 6a and fixed in a state where a tensile stress is applied to the sensing portion 45a. In a preferred mode, the tensile stress is applied along the expansion and contraction direction of the detection portions. When the sensor sheet is fixed to the fixing member in a state where a tensile stress is applied to the sensing portion, a tensile deformation state can be a reference state. Thus, suppression of zero drift of the strain sensor and measurement of the motion in the expansion and contraction direction are possible.

The tensile stress can be preferably greater than or equal to 0.003 N/mm and less than or equal to 0.08 N/mm, more preferably greater than or equal to 0.005 N/mm and less than or equal to 0.06 N/mm, and further preferably greater than or equal to 0.010 N/mm and less than or equal to 0.05 N/mm. By setting the tensile stress in the above-described range, zero drift can be more effectively suppressed.

The strain sensor 100a of the first embodiment configured as described above includes the fixing member, so, even when there is a soft intervening substance like a skin between a measuring object and the strain sensor, the degree to which the motion of the measuring object is absorbed by the intervening substance is equalized, and strain measurement with reduced variations is possible. The strain sensor 100a has a high mechanical strength and is easy to handle. In the strain sensor 100a, the sensor sheet 41a is fixed to the fixing member 6a in a state where a tensile stress is applied to the sensing portion 45a, so zero drift is suppressed.

### Second Embodiment

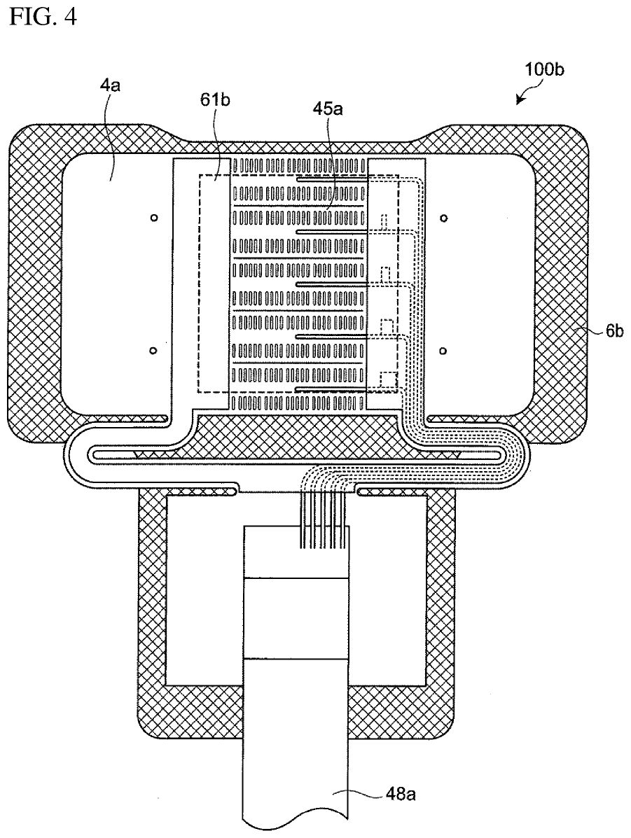

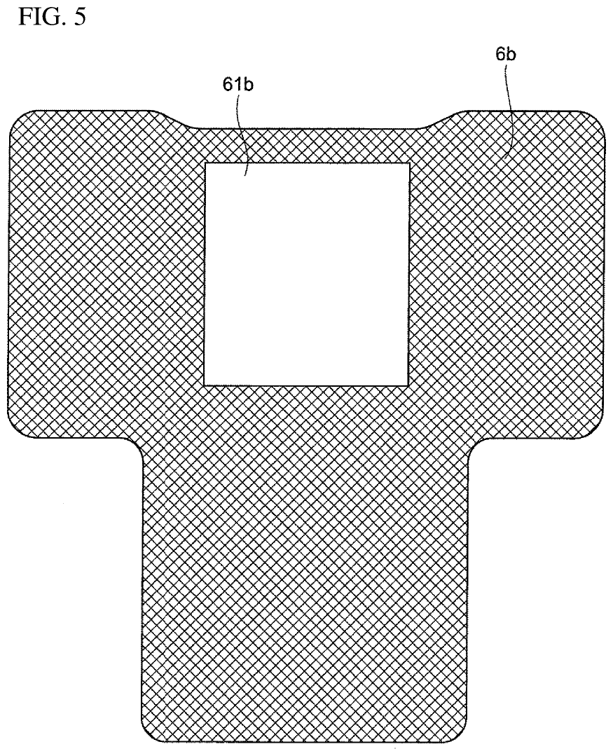

As shown in FIG. 4 and FIG. 5, a strain sensor 100b of a second embodiment has a similar configuration to that of the strain sensor 100a of the first embodiment except that the fixing member 6a is replaced with a fixing member 6b.

The fixing member 6b has a window 61b.

In the strain sensor 100b, the sensing portion 45a of the sensor sheet 41a is disposed so as to overlap the window 61b of the fixing member 6b. In other words, the fixing member 6b is present so as to surround the sensing portion 45a of the sensor sheet 41a in plan view.

In the strain sensor 100b, the outer shape of the strain sensor 100b is fixed by the fixing member 6b, so the strain sensor 100b has a certain mechanical strength, and occurrence of wrinkles in the sensing portion 45a is suppressed. On the other hand, in the strain sensor 100b, the sensing portion 45a is able to directly contact with an object to be measured, so the motion is detected with further high sensitivity.

### Third Embodiment

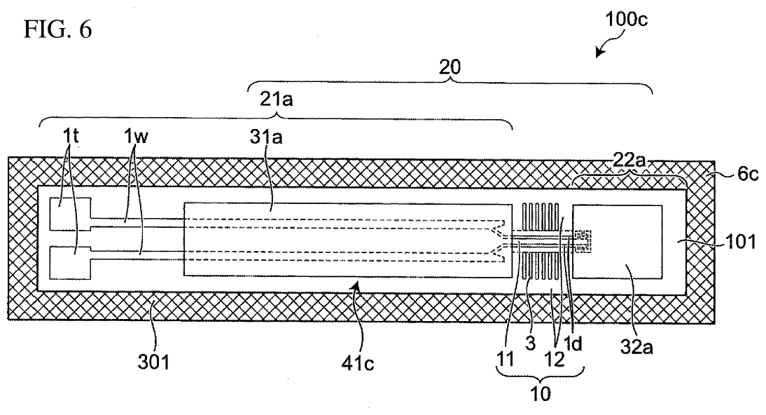

As shown in FIG. 6, a strain sensor 100c of a third embodiment includes a sensor sheet 41c and a fixing member 6c. The sensor sheet 41c is attached to the first main surface of the fixing member 6c. The strain sensor 100c of the third embodiment has a single detection portion.

The sensor sheet 41c included in the strain sensor 100c of the third embodiment is a strain sensor provided with a non-sensing portion 20 and a stretchable sensing portion 10 supported by the non-sensing portion 20. As shown in FIG. 6, in the sensor sheet 41c, the non-sensing portion 20 includes a first non-sensing portion 21a and a second non-sensing portion 22a. The sensing portion 10 is disposed between the first non-sensing portion 21a and the second non-sensing portion 22a.

The sensor sheet 41c includes a substrate 101 having a first main surface and a second main surface opposite to the first main surface, and a conductor portion provided on the first main surface of the substrate 101.

The conductor portion includes two connection terminal conductors 1t provided at a position spaced away from the sensing portion 10 on the first main surface of the first non-sensing portion 21a, two wiring conductors 1w respectively extending from the connection terminal conductors 1t in the same direction (hereinafter, referred to as first direction), and detection conductors 1d made up of two conductors respectively extending from the distal end portions of the wiring conductors 1w in the first direction and narrower than the wiring conductors 1w. Here, in the strain sensor 100c of the third embodiment, the two connection terminal conductors 1t, the two wiring conductors 1w, and the two detection conductors 1d are disposed symmetrically with respect to the center line in the first direction. The two connection terminal conductors 1t and the two wiring conductors 1w are provided on the first main surface of the first non-sensing portion 21a. The detection conductors 1d are provided on the first main surface of the sensing portion 10. A connection conductor that connects the distal end portions of the detection conductors 1d is provided on the first main surface of the second non-sensing portion 22a. As described above, a detection circuit in which the two detection conductors 1d are connected in series between the two connection terminal conductors 1t is constructed. In the detection circuit, the length of each of the detection conductors 1d in the first direction (expansion and contraction direction) changes according to expansion and contraction of the substrate of the sensing portion 10 or the resistance value of each of the detection conductors 1d changes with a change in cross sectional area. When a change in the resistance value of each of the detection conductors 1d is detected by, for example, a change in current value between the two connection terminal conductors 1t, the amount of expansion and contraction of the substrate 101 of the sensing portion 10, that is, a strain is detected. In other words, the detection conductors 1d make up a detection portion 11.

The sensing portion 10 is a region to measure a change in the shape of an object to be measured. The outer shape of the sensing portion 10 is set in consideration of the range of a measurement region. The followability of the sensing portion 10 is set in consideration of the elasticity of the object to be measured. As for the followability, for example, the substrate 101 of the sensing portion 10 has slits or holes and the thickness of the substrate is reduced to have a shape and structure easier to deform than the surroundings, thus enhancing the followability of the sensing portion 10.

As shown in FIG. 6, in the sensor sheet 41c, the sensing portion 10 includes the detection portion 11 made up of the detection conductors 1d, and low-elastic modulus portions 12 configured not to restrain a deformation to a strain in the detection portion 11 and not to restrain a deformation of an object to be measured. Specifically, in the sensor sheet 41c, the detection portion 11 is constructed with a narrow width so as not to restrain expansion and contraction of an object to be measured according to expansion and contraction of an object to be measured and so as to elastically deform following a strain of an object to be measured. In the sensor sheet 41c, the detection portion 11 is constructed such that the length in the first direction is greater than the width in a direction perpendicular to the first direction (that is, the width of each detection conductor 1d). Specifically, the detection portion 11 is preferably made up of the two detection conductors 1d parallel to the first direction, and the two detection conductors 1d are preferably juxtaposed in proximity to each other. By forming the detection portion 11 in this way, the rate of expansion and contraction in the expansion and contraction direction of the detection conductors 1d can be increased. The low-elastic modulus portions are preferably lower in elastic modulus than a measurement region of an object to be measured and easy to deform. The elastic modulus of the low-elastic modulus portions is preferably lower than or equal to a half and further preferably lower than or equal to one third of the elastic modulus of a measurement region of an object to be measured.

The low-elastic modulus portions 12 are respectively provided on both sides of the detection portion 11. The low-elastic modulus portions 12 each include a plurality of slits 3 provided in a direction that intersects with the expansion and contraction direction of the detection conductors 1d, preferably, provided in a perpendicular direction. Thus, the low-elastic modulus portions 12 expand and contract according to expansion and contraction of an object to be measured without restraining expansion and contraction of the object to be measured or expansion and contraction of the detection portion 11. With the thus configured sensing portion 10, the entire sensing portion 10 is able to deform according to a change in the shape of an object to be measured, without restraining a change in the shape of an object to be measured, for example, a bulge or the like on the skin of a human body. Therefore, by detecting expansion and contraction resulting from a change in the shape of an object to be measured with the detection portion 11, a strain in a measurement region of the object to be measured is detected.

As for the slit length of each slit 3 (the length of each slit in the expansion and contraction direction; here, the length in direction perpendicular to the first direction) formed in each low-elastic modulus portion 12, the total length of the slit lengths (total slit length) of the two slits 3 formed in the direction perpendicular to the first direction is preferably set so as to be greater than or equal to 40% and preferably greater than or equal to 60% of the width of the sensing portion 10. When the slits are formed such that the total slit length is greater than or equal to 40%, the same amount of strain is obtained by about two thirds of tensile load as compared to when no slit is formed. When the slits are formed such that the total slit length is greater than or equal to 60%, the same amount of strain is obtained by about half of tensile load as compared to when no slit is formed.

The non-sensing portion 20 is to, for example, fix the entire strain sensor by attaching the second main surface of the substrate 101 to the surface of an object to be measured, and supports the sensing portion 10 such that, when a measurement region of the object to be measured expands and contracts, the sensing portion 10 expands and contracts according to the expansion and contraction. In the sensor sheet 41c, the non-sensing portion 20 includes the first non-sensing portion 21a and the second non-sensing portion 22a. The first non-sensing portion 21a and the second non-sensing portion 22a are respectively provided on both sides of the sensing portion 10 in the expansion and contraction direction of the detection conductors 1d. The non-sensing portion 20 preferably includes restraint portions such that, when a measurement region in an object to be measured expands and contracts, a strain according to the expansion and contraction in the measurement region is detected under no influence of expansion and contraction in a region other than the measurement region.

As shown in FIG. 6, the restraint portions include, for example, a first restraint portion 31a provided in the first non-sensing portion 21a and a second restraint portion 32a provided in the second non-sensing portion 22a. The first restraint portion 31a and the second restraint portion 32a are preferably provided in proximity to the sensing portion 10. Thus, an influence of a portion other than a measurement region of an object to be measured is reduced, with the result that a strain in the measurement region can be accurately measured.

The fixing member 6c is a sheet-shaped member having a first main surface and a second main surface opposite to the first main surface. The fixing member 6c has a similar configuration to that of the fixing member 6a of the strain sensor 100a of the first embodiment except that the shape in plan view is a shape along the shape of the sensor sheet 41c.

The strain sensor 100c of the third embodiment includes the sensor sheet 41c and the fixing member 6c. The sensor sheet 41c is fixed so as to overlap the fixing member 6c. The strain sensor 100c is particularly capable of detecting a strain in the first direction.

As for the strain sensor 100c, the configuration and features other than the above can be similar to those of the strain sensor 100a of the first embodiment.

The strain sensor 100c of the third embodiment is simple in structure, easy to manufacture, and easy to handle.

### Fourth Embodiment

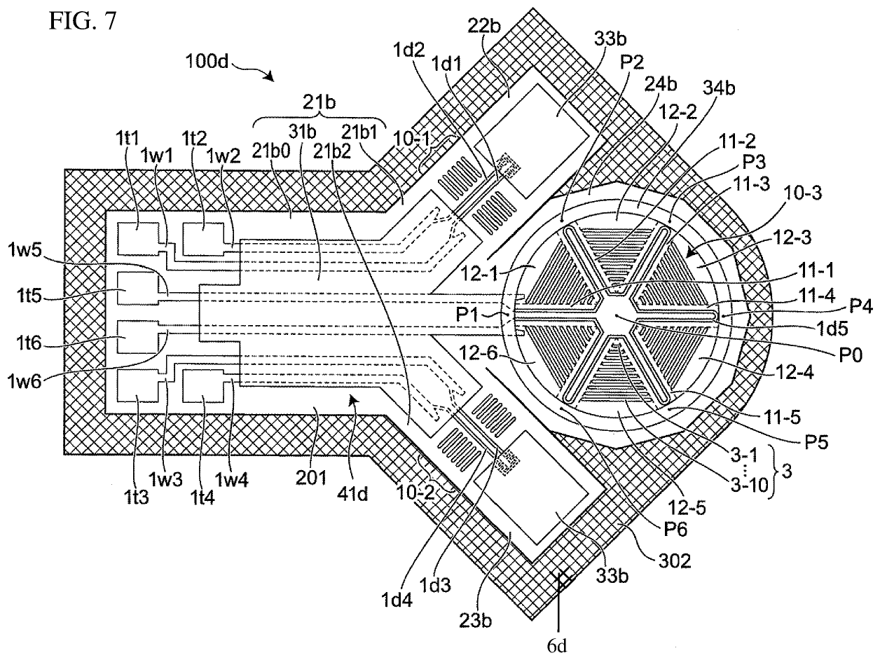

As shown in FIG. 7, a strain sensor 100d of a fourth embodiment includes a sensor sheet 41d and a fixing member 6d. The sensor sheet 41d is attached to the first main surface of the fixing member 6d. The strain sensor 100d of the fourth embodiment has a plurality of detection portions.

As shown in FIG. 7, the sensor sheet 41d includes three sensing portions, that is, a first sensing portion 10-1, a second sensing portion 10-2, and a third sensing portion 10-3. Here, the first sensing portion 10-1 and the second sensing portion 10-2 are provided so as to mainly detect a strain due to two-dimensional expansion and contraction, and the third sensing portion 10-3 is provided so as to mainly detect a strain due to three-dimensional expansion and contraction.

The sensor sheet 41d includes a first non-sensing portion 21b, a second non-sensing portion 22b, a third non-sensing portion 23b, and a fourth non-sensing portion 24b as non-sensing portions. Here, the first non-sensing portion 21b includes a base non-sensing portion 21b0, a first branch non-sensing portion 21b1 extending from the base non-sensing portion 21b0 in a first direction, and a second branch non-sensing portion 21b2 extending from the base non-sensing portion 21b0 in a second direction perpendicular to the first direction. The fourth non-sensing portion 24b is provided in an annular shape.

The first sensing portion 10-1 is provided between the first branch non-sensing portion 21b1 and the second non-sensing portion 22b. The second sensing portion 10-2 is provided between the second branch non-sensing portion 21b2 and the third non-sensing portion 23b. The third sensing portion 10-3 is provided inside the annular fourth non-sensing portion 24b. Here, the third sensing portion 10-3 provided inside the fourth non-sensing portion 24b is configured as will be described in detail later and mainly detects a strain in a direction perpendicular to the first direction and the second direction, that is, a height direction.

The sensor sheet 41d includes a substrate 201 having a first main surface and a second main surface opposite to the first main surface, and a conductor portion provided on the first main surface of the substrate 201.

The substrate 201 has a base portion corresponding to the base non-sensing portion 21b0, a first branch portion extending from the base portion in the first direction, a second branch portion extending from the base portion in the second direction perpendicular to the first direction, and a substantially circular-shaped circular portion placed between the first branch portion and the second branch portion. In the second embodiment, the circular portion is provided such that the center is located on the central axis of the base portion. The first branch non-sensing portion 21b1, the first sensing portion 10-1, and the second non-sensing portion 22b are provided in the first branch portion. The second branch non-sensing portion 21b2, the second sensing portion 10-2, and the third non-sensing portion 23b are provided in the second branch portion. The fourth non-sensing portion 24b and the third sensing portion 10-3 are provided in the circular portion.

The conductor portion has six first to sixth connection terminal conductors 1t1 to 1t6 on the first main surface of the base non-sensing portion 21b0 (the base portion of the substrate 201). The first to sixth connection terminal conductors 1t1 to 1t6 are provided at a position across from the first branch non-sensing portion 21b1 and the second branch non-sensing portion 21b2 on the first main surface of the base non-sensing portion 21b0.

The conductor portion has first to six wiring conductors 1w1 to 1w6 respectively extending from the first to sixth connection terminal conductors 1t1 to 1t6. The first and second wiring conductors 1w1, 1w2 are provided parallel and adjacent to each other and provided so as to extend from the base non-sensing portion 21b0 to the first branch non-sensing portion 21b1. The third and fourth wiring conductors 1w3, 1w4 are provided parallel and adjacent to each other and provided so as to extend from the base non-sensing portion 21b0 to the second branch non-sensing portion 21b2. The fifth and sixth wiring conductors 1w5, 1w6 are provided parallel and adjacent to each other and provided so as to extend from the base non-sensing portion 21b0 to the fourth non-sensing portion 24b.

The conductor portion further has first to fifth detection conductors 1d1 to 1d5 respectively extending from the distal end portions of the first to sixth wiring conductors 1w1 to 1w6. The first to fifth detection conductors 1d1 to 1d5 are respectively formed with widths less than the widths of the first to sixth wiring conductors 1w1 to 1w6. The first and second detection conductors 1d1, 1d2 are provided in the first sensing portion 10-1, and the distal end portions of the first and second detection conductors 1d1, 1d2 are connected in the second non-sensing portion 22b. The third and fourth detection conductors 1d3, 1d4 are provided in the second sensing portion 10-2, and the distal end portions of the third and fourth detection conductors 1d3, 1d4 are connected in the third non-sensing portion 23b.

One end of the fifth detection conductor 1d5 is connected to the fifth wiring conductor 1w5, and the other end of the fifth detection conductor 1d5 is connected to the sixth wiring conductor 1w6. As will be described in detail later, the fifth detection conductor 1d5 is provided in the third sensing portion 10-3. The constituent material of the conductor portion is similar to that of the strain sensor of the first embodiment.

In this way, a first detection circuit in which the first and second detection conductors 1d1, 1d2 are connected in series between the first and second connection terminal conductors 1t1, 1t2 is constructed. In the first detection circuit, the first and second detection conductors 1d1, 1d2 change in length in the first direction according to expansion and contraction of the substrate of the first sensing portion 10-1, with the result that the resistance values of the first and second detection conductors 1d1, 1d2 change. When the change in the resistance value of each of the first and second detection conductors 1d1, 1d2 is detected in accordance with, for example, a change in current value between the first and second connection terminal conductors 1t1, 1t2, the amount of expansion and contraction, that is, strain, of the substrate of the first sensing portion 10-1 is detected. In other words, the first and second detection conductors 1d1, 1d2 make up one detection portion.

A second detection circuit in which the third and fourth detection conductors 1d3, 1d4 are connected in series between the third and fourth connection terminal conductors 1t3, 1t4 is constructed. In the second detection circuit, the third and fourth detection conductors 1d3, 1d4 change in length in the second direction according to expansion and contraction of the substrate of the second sensing portion 10-2, with the result that the resistance values of the third and fourth detection conductors 1d3, 1d4 change. When the change in the resistance value of each of the third and fourth detection conductors 1d3, 1d4 is detected in accordance with, for example, a change in current value between the third and fourth connection terminal conductors 1t3, 1t4, the amount of expansion and contraction, that is, strain, of the substrate of the second sensing portion 10-2 is detected. In other words, the third and fourth detection conductors 1d3, 1d4 make up one detection portion.

In the sensor sheet 41d, the configuration of each of the first sensing portion 10-1 and the second sensing portion 10-2 is similar to that of the sensing portion 10 in the strain sensor of the third embodiment. Therefore, hereinafter, the configuration of the third sensing portion 10-3, different from that of the third embodiment, will be mainly described.

The third sensing portion 10-3 is provided inside the annular fourth non-sensing portion 24b and, as described above, mainly detects a strain in the direction perpendicular to the first direction and the second direction.

The third sensing portion 10-3 is a region to measure a change in the shape of an object to be measured and located inside the annular fourth non-sensing portion 24b. The third sensing portion 10-3 includes a plurality of (six) sector low-elastic modulus portions 12-1 to 12-6, and a plurality of (six) detection portions 11-1 to 11-6 each located between any adjacent two of the low-elastic modulus portions and extending radially from the center of the third sensing portion 10-3. Each of the detection portions 11-1 to 11-6 is formed such that the length in the radial direction of the third sensing portion 10-3 is longer than the width in a direction perpendicular to the radial direction. Thus, the detection portions 11-1 to 11-6 are elastically deformed without restraining expansion and contraction of an object to be measured according to the expansion and contraction of the object to be measured. Here, the expansion and contraction direction of each of the detection portions 11-1 to 11-6 is the length direction of the detection conductor that makes up the detection portion, that is, a direction connecting point P0 to an associated one of point P1 to point P6. One end of the fifth detection conductor 1d5 is connected to the fifth wiring conductor 1w5, extended to the detection portion 11-1, routed in a meandering manner in each of the detection portions 11-2 to 11-6, and then the other end of the fifth detection conductor 1d5 is connected to the sixth wiring conductor 1w6 via the detection portion 11-1.

A plurality of low-elastic modulus portions 12-1 to 12-6 each has, for example, a plurality of, that is, 10 slits 3-1 to 3-10. In each of the low-elastic modulus portions, the plurality of slits 3-1 to 3-10 is formed such that the centers of the slits 3-1 to 3-10 are located on the center line that bisects the central angle of the sector and the expansion and contraction direction is perpendicular to the center line. In addition, in each of the low-elastic modulus portions 12-1 to 12-6, the plurality of slits 3-1 to 3-10 is formed such that the slit length (the length in the direction perpendicular to the center line) increases with the distance from the center of the sector. Thus, the low-elastic modulus portions 12-1 to 12-6 expand and contract without restricting expansion and contraction of an object to be measured or expansion and contraction of each of the detection portions 11-1 to 11-6 according to the expansion and contraction of the object to be measured. Preferably, the plurality of slits 3-1 to 3-10 is preferably formed such that the interval between an end portion of each of the plurality of slits 3-1 to 3-10 and the fifth wiring conductor 1w5 proximate to the end portion is equal. In the present embodiment, the third sensing portion 10-3 is configured to include the six low-elastic modulus portions. The third sensing portion 10-3 just needs to include at least two or more low-elastic modulus portions. The slits are not limited to a linear slit and may be formed in a circular arc shape.

The fourth non-sensing portion 24b is provided in an annular shape around the third sensing portion 10-3 and fixes the surroundings of the third sensing portion 10-3 when the second main surface of the substrate 201 in the fourth non-sensing portion 24b is attached to the surface of an object to be measured. The fourth non-sensing portion 24b supports the third sensing portion 10-3 such that, when a measurement region of the object to be measured expands and contracts, the third sensing portion 10-3 expands and contracts according to the expansion and contraction. The fourth non-sensing portion 24b preferably includes a restraint portion 34b such that, when a measurement region in an object to be measured expands and contracts, a strain according to the expansion and contraction in the measurement region is detected under no influence of expansion and contraction in a region other than the measurement region. As shown in FIG. 7, the restraint portion 34b is preferably provided around the third sensing portion 10-3 and preferably provided in proximity to the third sensing portion 10-3. Thus, an influence of a portion other than a measurement region of an object to be measured is reduced, with the result that a strain in the measurement region can be accurately measured.

The fixing member 6d is a sheet-shaped member having a first main surface and a second main surface opposite to the first main surface. The fixing member 6d has a similar configuration to that of the fixing member 6a of the strain sensor 100a of the first embodiment except that, in plan view, the fixing member 6d has a shape that overlaps the entire sensor sheet 41d.

As for the strain sensor 100d, the configuration and features other than the above can be similar to the strain sensor 100a of the first embodiment.

The strain sensor 100d of the fourth embodiment configured as described above includes the first sensing portion 10-1, the second sensing portion 10-2, and the third sensing portion 10-3 that are capable of expanding and contracting according to a strain, so the strain sensor 100d is capable of detecting a strain in a small deformation area, for example, a bulge or the like of a skin of a human body.

In the strain sensor 100d of the fourth embodiment configured as described above, the first sensing portion 10-1 has a high sensitivity to expansion and contraction in the first direction, the second sensing portion 10-2 has a high sensitivity to expansion and contraction in the second direction, and each of the detection portions of the third sensing portion 10-3 expands and contracts in an associated one of P0-P1 direction, P0-P2 direction, P0-P3 direction, P0-P4 direction, P0-P5 direction, and P0-P6 direction and has a high sensitivity to expansion and contraction in the direction perpendicular to the first direction and the second direction, that is, the direction perpendicular to the first main surface of the substrate 201. Disposition of the first sensing portion 10-1 and the second sensing portion 10-2 is not limited to a position in which the first sensing portion 10-1 and the second sensing portion 10-2 are perpendicular to each other. The strain sensor 100d is attached such that the first sensing portion 10-1, the second sensing portion 10-2, and the third sensing portion 10-3 are appropriately disposed in accordance with a main expansion and contraction direction of a measurement region of an object to be measured, and is able to measure a strain with high sensitivity in each measurement portion. With this configuration, a strain of an object to be measured in each of three X, Y, and Z directions can be detected, so the shape of a deformation causing a strain can be estimated from all of these strains.

### Fifth Embodiment

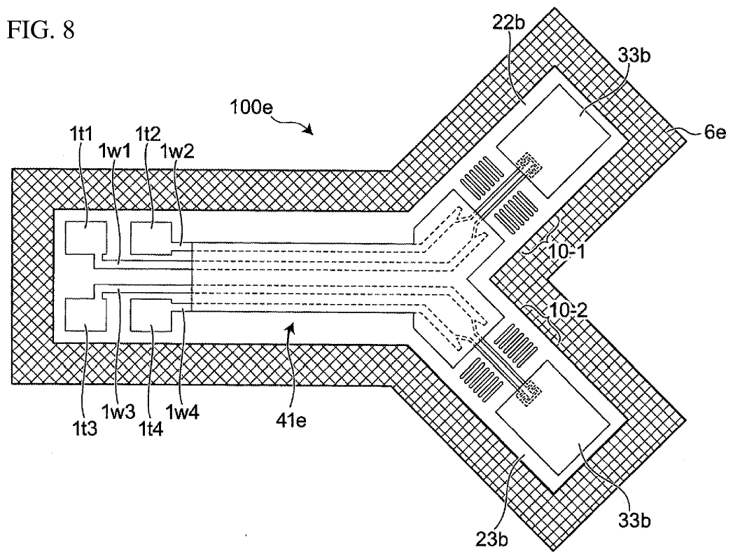

As shown in FIG. 8, a strain sensor 100e of a fifth embodiment includes a sensor sheet 41e and a fixing member 6e. The sensor sheet 41e is attached to the first main surface of the fixing member 6e. The strain sensor 100e of the fifth embodiment has a configuration excluding the third sensing portion 10-3 from the strain sensor 100d of the fourth embodiment. In other words, the strain sensor 100e of the fifth embodiment is a modification of the strain sensor 100d of the fourth embodiment.

With the strain sensor 100e, a strain sensor with a high sensitivity to expansion and contraction in the first direction and the second direction is provided at lower cost than the strain sensor of the fourth embodiment.

### Sixth Embodiment

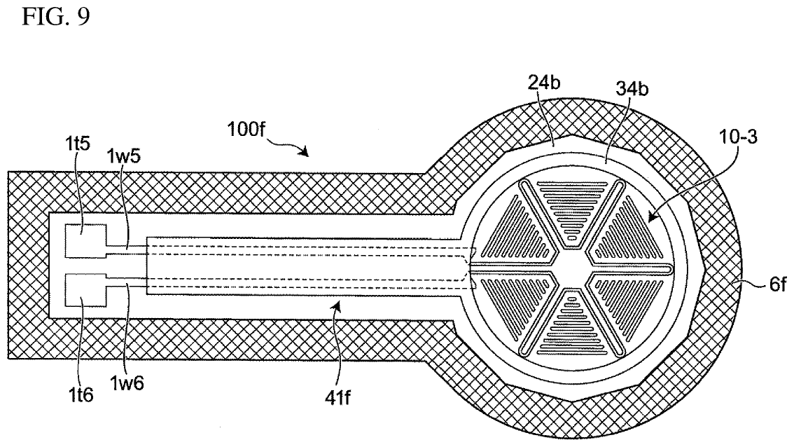

As shown in FIG. 9, a strain sensor 100f of a sixth embodiment includes a sensor sheet 41f and a fixing member 6f. The sensor sheet 41f is attached to the first main surface of the fixing member 6f. The strain sensor 100f of the sixth embodiment has a configuration excluding the first sensing portion 10-1 and the second sensing portion 10-2 from the strain sensor 100d of the fourth embodiment. In other words, the strain sensor 100f of the sixth embodiment is a modification of the strain sensor 100d of the fourth embodiment.

With the strain sensor 100f, a strain sensor with a high sensitivity in the direction perpendicular to the first direction and the second direction is provided at lower cost than the strain sensor of the fourth embodiment.

### Seventh Embodiment

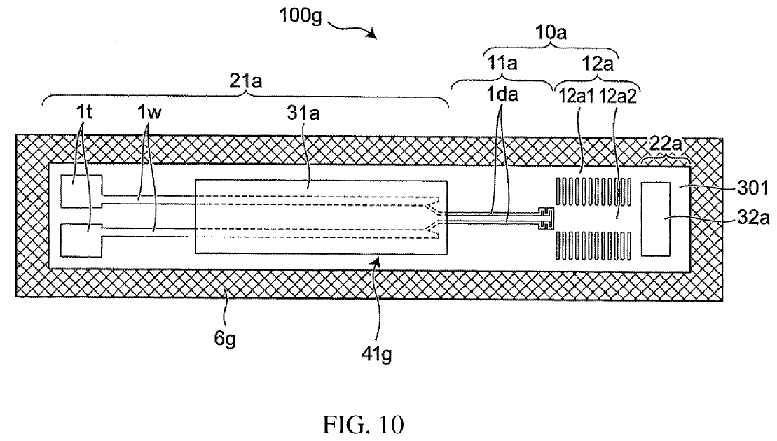

As shown in FIG. 10, a strain sensor 100g of a seventh embodiment includes a sensor sheet 41g and a fixing member 6g. The sensor sheet 41g is attached to the first main surface of the fixing member 6g. The strain sensor 100g of the seventh embodiment has a similar configuration to that of the strain sensor 100c of the third embodiment except that the configuration of a sensing portion 10a of the sensor sheet 41g is different.

The sensing portion 10a in the sensor sheet 41g is suitable for a case where a strain resulting from a large deformation due to a force causing the strain is detected as compared to the strain sensor of the third embodiment. Specifically, in the sensor sheet 41g of the seventh embodiment, as shown in FIG. 10, the sensing portion 10a includes a detection portion 11a made up of a detection conductor 1da, and a low-elastic modulus portion 12a. The low-elastic modulus portion 12a is disposed between the detection portion 11a and the second non-sensing portion 22a.

In the sensor sheet 41g, the low-elastic modulus portion 12a includes a first low-elastic modulus portion 12a1 and a second low-elastic modulus portion 12a2 disposed symmetrically with respect to the center line in a first direction that is an extension direction of the detection conductor 1da. Each of the first low-elastic modulus portion 12a1 and the second low-elastic modulus portion 12a2 includes a plurality of slits of which the length in a direction perpendicular to the first direction is greater than the width in the first direction. The thus configured low-elastic modulus portion 12a (the first low-elastic modulus portion 12a1 and the second low-elastic modulus portion 12a2) has a higher rate of expansion and contraction in the first direction than the detection portion 11a.

In the sensing portion 10a of the sensor sheet 41g configured as described above, when the entire sensing portion 10a receives a large deformation, the low-elastic modulus portion 12a with a higher rate of expansion and contraction than the detection portion 11a deforms by a large amount, with the result that a break of the detection conductor 1da formed in the detection portion 11a is prevented. The detection portion 11a can be formed in a wider width than the detection portion 11 of the sensor sheet 41c in the strain sensor 100c of the third embodiment, so a break of the detection conductor 1da is further effectively prevented. In this way, in the sensor sheet 41g, the low-elastic modulus portion 12a capable of elastically deforming by a large amount is disposed between the detection portion 11a and the second non-sensing portion 22a. Therefore, when a large deformation occurs in the sensing portion 10a, a strain is detected without a break of the detection conductor 1da.

In addition, the sensor sheet 41g includes the first restraint portion 31a and the second restraint portion 32a. Therefore, an influence of a portion other than a measurement region of an object to be measured is reduced, with the result that a strain in the measurement region can be accurately measured.

In the strain sensors of the fourth and sixth embodiments, the first sensing portion 10-1 and/or the second sensing portion 10-2 may be configured similarly to the sensing portion 10a of the seventh embodiment.

As for the strain sensor 100g, the configuration and features other than the above can be similar to the strain sensor 100a of the first embodiment.

### Eighth Embodiment

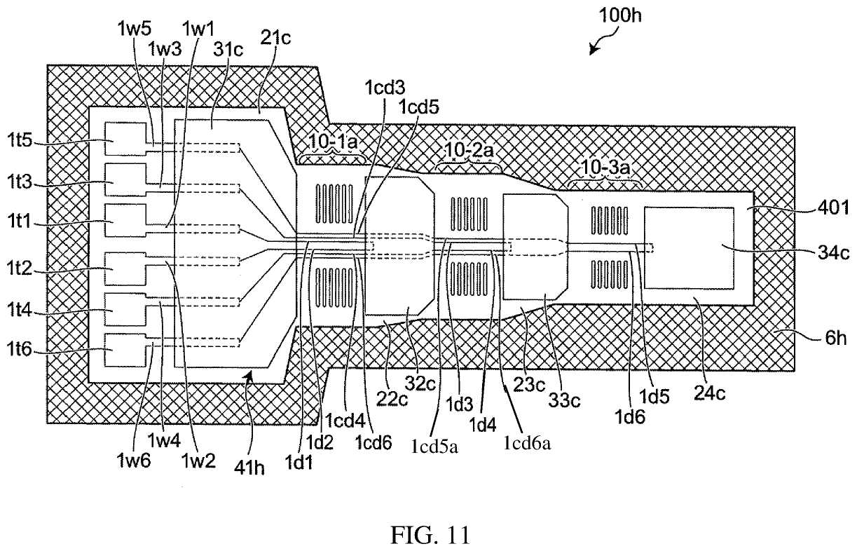

As shown in FIG. 11, a strain sensor 100h of an eighth embodiment includes a sensor sheet 41h and a fixing member 6h. The sensor sheet 41h is attached to the first main surface of the fixing member 6h.

As shown in FIG. 11, the sensor sheet 41h is a strain sensor in which a non-sensing portion and a sensing portion are alternately provided in a first direction, and includes four non-sensing portions, that is, a first non-sensing portion 21c, a second non-sensing portion 22c, a third non-sensing portion 23c, and a fourth non-sensing portion 24c, and three sensing portions, that is, a first sensing portion 10-1a, a second sensing portion 10-2a, and a third sensing portion 10-3a. In the sensor sheet 41h, the first sensing portion 10-1a is provided between the first non-sensing portion 21c and the second non-sensing portion 22c, the second sensing portion 10-2a is provided between the second non-sensing portion 22c and the third non-sensing portion 23c, and the third sensing portion 10-3a is provided between the third non-sensing portion 23c and the fourth non-sensing portion 24c.

In the sensor sheet 41h, the first non-sensing portion 21c includes first to sixth connection terminal conductors 1t1 to 1t6. Here, the first and second connection terminal conductors 1t1, 1t2 are provided on the inner side closest to the center line extending in the first direction, the third and fourth connection terminal conductors 1t3, 1t4 are provided on the outer sides of the first and second connection terminal conductors 1t1, 1t2, and the fifth and sixth connection terminal conductors 1t5, 1t6 are provided on the outermost sides. In the first non-sensing portion 21c, first to sixth wiring conductors 1w1 to 1w6 are respectively extended from the first to sixth connection terminal conductors 1t1 to 1t6 in the first direction, and the distal ends of the first to sixth wiring conductors 1w1 to 1w6 are gathered near the center line and routed so as to be proximate to one another in a state of being separated from one another at the boundary between the first non-sensing portion 21c and the first sensing portion 10-1a.

First and second detection conductors 1d1, 1d2 that detect a strain of the first sensing portion 10-1a are provided between the first and second connection terminal conductors 1t1, 1t2, third and fourth detection conductors 1d3, 1d4 that detect a strain of the second sensing portion 10-2a are provided between the third and fourth connection terminal conductors 1t3, 1t4, and fifth and sixth detection conductors 1d5, 1d6 that detect a strain of the third sensing portion 10-3a are provided between the fifth and sixth connection terminal conductors 1t5, 1t6, as described below.

The first and second detection conductors 1d1, 1d2 respectively extend from the distal ends of the first and second wiring conductors 1w1, 1w2 and provided in the first sensing portion 10-1a, and the distal end portions of the first and second detection conductors 1d1, 1d2 are connected in the second non-sensing portion 22c. The third detection conductor 1d3 is provided in the second sensing portion 10-2a via a third conductor 1cd3 and a connection conductor. The third conductor 1cd3 is extended from the distal end of the third wiring conductor 1w3 and provided in the first sensing portion 10-1a. The connection conductor is extended from the distal end of the third conductor 1cd3 and provided in the second non-sensing portion 22c. The fourth detection conductor 1d4 is provided in the second sensing portion 10-2a via a fourth conductor 1cd4 and a connection conductor. The fourth conductor 1cd4 is extended from the distal end of the fourth wiring conductor 1w4 and provided in the first sensing portion 10-1a. The connection conductor is extended from the distal end of the fourth conductor 1cd4 and provided in the second non-sensing portion 22c. The distal end portion of the third detection conductor 1d3 and the distal end portion of the fourth detection conductor 1d4 are connected in the third non-sensing portion 23c.

The fifth detection conductor 1d5 is provided in the third sensing portion 10-3a via a fifth conductor 1cd5, a connection conductor, a fifth conductor 1cd5a, and another connection conductor. The fifth conductor 1cd5 is extended from the distal end of the fifth wiring conductor 1w5 and provided in the first sensing portion 10-1a. The connection conductor is extended from the distal end of the fifth conductor 1cd5 and provided in the second non-sensing portion 22c. The fifth conductor 1cd5a is extended from the distal end of the connection conductor and provided in the second sensing portion 10-2a. The other connection conductor is extended from the distal end of the fifth conductor 1cd5a and provided in the third non-sensing portion 23c.

The sixth detection conductor 1d6 is provided in the third sensing portion 10-3a via a sixth conductor 1cd6, a connection conductor, a sixth conductor 1cd6a, and another connection conductor. The sixth conductor 1cd6 is extended from the distal end of the sixth wiring conductor 1w6 and provided in the first sensing portion 10-1a. The connection conductor is extended from the distal end of the sixth conductor 1cd6 and provided in the second non-sensing portion 22c. The sixth conductor 1cd6a is extended from the distal end of the connection conductor and provided in the second sensing portion 10-2a. The other connection conductor is extended from the distal end of the sixth conductor 1cd6a and provided in the third non-sensing portion 23c.

The distal end portion of the fifth detection conductor 1d5 and the distal end portion of the sixth detection conductor 1d6 are connected in the fourth non-sensing portion 24c. Here, the resistance values of the connection conductors formed in the non-sensing portions do not substantially change due to a strain.

As described above, a first detection circuit is constructed between the first and second connection terminal conductors 1t1, 1t2. The first detection circuit in which the first detection conductor 1d1 and the second detection conductor 1d2 are connected in series is used to detect a strain of the first sensing portion 10-1a.

A second detection circuit is constructed between the third and fourth connection terminal conductors 1t3, 1t4. The second detection circuit in which the third conductor 1cd3, the third detection conductor 1d3, the fourth detection conductor 1d4, and the fourth conductor 1cd4 are connected in series is used to detect a strain of the second sensing portion 10-2a.

A third detection circuit is constructed between the fifth and sixth connection terminal conductors 1t5, 1t6. The third detection circuit in which the fifth conductor 1cd5, the fifth conductor 1cd5a, the fifth detection conductor 1d5, the sixth detection conductor 1d6, the sixth conductor 1cd6a, and the sixth conductor 1cd6 are connected in series is used to detect a strain of the third sensing portion 10-3a.

Here, in the first detection circuit, since a change in resistance value between the first and second connection terminal conductors 1t1, 1t2 is a change in resistance value between the first detection conductor 1d1 and the second detection conductor 1d2, a strain in the first sensing portion 10-1a is detected in accordance with a change in resistance value between the first and second connection terminal conductors 1t1, 1t2.

However, the second detection circuit and the third detection circuit include the third conductor 1cd3, the fourth conductor 1cd4, the fifth conductor 1cd5, the fifth conductor 1cd5a, the sixth conductor 1cd6a, and the sixth conductor 1cd6 that are formed in other sensing portions and that change in resistance value due to a strain of the other sensing portions in addition to the third detection conductor 1d3, the fourth detection conductor 1d4, the fifth detection conductor 1d5 and the sixth detection conductor 1d6 for detecting a strain of an associated one of the sensing portions.

Therefore, in the second detection circuit and the third detection circuit, except a change in resistance value in the conductors formed in the sensing portions other than the sensing portion on an object to be measured, a change in resistance value in the third detection conductor 1d3 and the fourth detection conductor 1d4 or a change in resistance value in the fifth detection conductor 1d5 and the sixth detection conductor 1d6, in the sensing portion on the object to be measured, needs to be calculated.

In the second detection circuit and the third detection circuit, various methods for excluding a change in resistance value in the conductors formed in the sensing portions other than the sensing portion on the object to be measured are conceivable, and, for example, the following method may be adopted.

For example, for the second detection circuit, the third conductor 1cd3 and the fourth conductor 1cd4 provided in the first sensing portion 10-1a have the same configuration as the first and second detection conductors 1d1, 1d2 of the first detection circuit. The same configuration means using the same material and constructing the third conductor 1cd3 and the fourth conductor 1cd4 in the same shape as the first and second detection conductors 1d1, 1d2. Thus, a change in the resistance value of each of the third conductor 1cd3 and the fourth conductor 1cd4 is substantially the same as a change in the resistance value of each of the first and second detection conductors 1d1, 1d2.

Therefore, by excluding a change in the resistance value of each of the first and second detection conductors 1d1, 1d2, detected in the first detection circuit, from a change in the resistance value of the second detection circuit between the third and fourth connection terminal conductors 1t3, 1t4, a change in the resistance value of each of the third detection conductor 1d3 and the fourth detection conductor 1d4 in the second detection circuit is calculated.

Similarly, for the third detection circuit, the fifth conductor 1cd5 and the sixth conductor 1cd6, provided in the first sensing portion 10-1a, just need to have the same configuration as the first and second detection conductors 1d1, 1d2 of the first detection circuit, and the fifth conductor 1cd5a and the sixth conductor 1cd6a, provided in the second sensing portion 10-2a, just need to have the same configuration as the third detection conductor 1d3 and the fourth detection conductor 1d4 in the second detection circuit. Thus, by excluding a change in the resistance value of each of the first and second detection conductors 1d1, 1d2, detected in the first detection circuit, and a change in the resistance value of each of the third detection conductor 1d3 and the fourth detection conductor 1d4 in the second detection circuit from a change in the resistance value of the third detection circuit between the fifth and sixth connection terminal conductors 1t5, 1t6, a change in the resistance value of each of the fifth detection conductor 1d5 and the sixth detection conductor 1d6 in the third detection circuit is calculated.

The strain sensor 100h of the eighth embodiment including the sensor sheet 41h configured as described above is capable of performing a differential measurement on strains of a plurality of detection regions for a relatively narrow object to be detected and is capable of, for example, detecting a bulge or swelling in a plurality of points for a finger of a human body.

In the strain sensor 100h of the eighth embodiment, the first non-sensing portion 21c, the second non-sensing portion 22c, the third non-sensing portion 23c, and the fourth non-sensing portion 24c preferably respectively include a first restraint portion 31c, a second restraint portion 32c, a third restraint portion 33c, and a fourth restraint portion 34c such that, when a measurement region in an object to be measured expands and contracts, a strain according to the expansion and contraction in the measurement region can be detected under no influence of extension and contraction in a region other than the measurement region.

As for the strain sensor 100h, the configuration and features other than the above can be similar to the strain sensor 100a of the first embodiment.

### Ninth Embodiment

As shown in FIG. 12, a strain sensor 100i of a ninth embodiment includes a sensor sheet 41i and a fixing member 6i. The sensor sheet 41i is attached to the first main surface of the fixing member 6i.

As shown in FIG. 12, in the sensor sheet 41i, a non-sensing portion and a measurement portion are alternately provided in a first direction. The sensor sheet 41i includes four non-sensing portions, that is, a first non-sensing portion 21d, a second non-sensing portion 22d, a third non-sensing portion 23d, and a fourth non-sensing portion 24d, and three sensing portions, that is, a first sensing portion 10-1b, a second sensing portion 10-2b, and a third sensing portion 10-3b. In the sensor sheet 41i, the first sensing portion 10-1b is provided between the first non-sensing portion 21d and the second non-sensing portion 22d, the second sensing portion 10-2b is provided between the second non-sensing portion 22d and the third non-sensing portion 23d, and the third sensing portion 10-3b is provided between the third non-sensing portion 23d and the fourth non-sensing portion 24d.

As described above, the sensor sheet 41i in which the non-sensing portion and the measurement portion are alternately provided in the first direction is similar to the sensor sheet 41h in the strain sensor 100h of the eighth embodiment; however, the shape of each of the three non-sensing portions, that is, the first non-sensing portion 21d, the second non-sensing portion 22d, and the third non-sensing portion 23d, other than the fourth non-sensing portion 24d, differs from the shape of each of the first to third non-sensing portions 21c, 22c, 23c of the eighth embodiment. Specifically, in the sensor sheet 41i, the first non-sensing portion 21d, the second non-sensing portion 22d, and the third non-sensing portion 23d respectively include a first wiring non-sensing portion 21dc, a second wiring non-sensing portion 22dc, and a third wiring non-sensing portion 23dc, each extending in a direction perpendicular to the first direction. In the following description, a portion in the first non-sensing portion 21d, other than the first wiring non-sensing portion 21dc, a portion in the second non-sensing portion 22d, other than the second wiring non-sensing portion 22dc, and a portion in the third non-sensing portion 23d, other than the third wiring non-sensing portion 23dc, are referred to as first measurement non-sensing portion 21dm, second measurement non-sensing portion 22dm, and third measurement non-sensing portion 23dm.

In the first non-sensing portion 21d, the first wiring non-sensing portion 21dc includes a first connection terminal conductor 1t1 and a second connection terminal conductor 1t2 at an end portion across from the first measurement non-sensing portion 21dm. In the first non-sensing portion 21d, first and second wiring conductors 1w1d, 1w2d are respectively extended from the first and second connection terminal conductors 1t1, 1t2 in a direction perpendicular to the first direction and then bent to the first direction in the first measurement non-sensing portion 21dm and routed.

The second wiring non-sensing portion 22dc includes a third connection terminal conductor 1t3 and a fourth connection terminal conductor 1t4 at an end portion across from the second measurement non-sensing portion 22dm. In the second non-sensing portion 22d, third and fourth wiring conductors 1w3d, 1w4d are respectively extended from the third and fourth connection terminal conductors 1t3, 1t4 in a direction perpendicular to the first direction and then bent to the first direction in the second measurement non-sensing portion 22dm and routed.

A fifth connection terminal conductor 1t5 and a sixth connection terminal conductor 1t6 are included in the third wiring non-sensing portion 23dc at an end portion across from the third measurement non-sensing portion 23dm. In the third non-sensing portion 23d, fifth and sixth wiring conductors 1w5d, 1w6d are respectively extended from the fifth and sixth connection terminal conductors 1t5, 1t6 in a direction perpendicular to the first direction and then bent to the first direction in the third measurement non-sensing portion 23dm and routed.

The first and second detection conductors 1d1, 1d2 respectively extend from the distal ends of the first and second wiring conductors 1w1d, 1w2d and provided in the first sensing portion 10-1b, and the distal end portions of the first and second detection conductors 1d1, 1d2 are connected in the second non-sensing portion 22d.

The third and fourth detection conductors 1d3, 1d4 respectively extend from the distal ends of the third and fourth wiring conductors 1w3d, 1w4d and provided in the second sensing portion 10-2b, and the distal end portions of the third and fourth detection conductors 1d3, 1d4 are connected in the third non-sensing portion 23d.

The fifth and sixth detection conductors 1d5, 1d6 respectively extend from the distal ends of the fifth and sixth wiring conductors 1w5d, 1w6d and provided in the third sensing portion 10-3b, and the distal end portions of the fifth and sixth detection conductors 1d5, 1d6 are connected in the fourth non-sensing portion 24d.

As described above, a first detection circuit is constructed between the first and second connection terminal conductors 1t1, 1t2. The first detection circuit in which the first and second detection conductors 1d1, 1d2 are connected in series is used to detect a strain of the first sensing portion 10-1d.

A second detection circuit is constructed between the third and fourth connection terminal conductors 1t3, 1t4. The second detection circuit in which the third and fourth detection conductors 1d3, 1d4 are connected in series is used to detect a strain of the second sensing portion 10-2d.

A third detection circuit is constructed between the fifth and sixth connection terminal conductors 1t5, 1t6. The third detection circuit in which the fifth and sixth detection conductors 1d5, 1d6 are connected in series is used to detect a strain of the third sensing portion 10-3d.

The sensor sheet 41i configured as described above is capable of performing differential measurement on strains of a plurality of detection regions.

In the sensor sheet 41i, the first non-sensing portion 21d, the second non-sensing portion 22d, the third non-sensing portion 23d, and the fourth non-sensing portion 24d preferably respectively include a first restraint portion 31d, a second restraint portion 32d, a third restraint portion 33d, and a fourth restraint portion 34d such that, when a measurement region in an object to be measured expands and contracts, a strain according to the expansion and contraction in the measurement region can be detected under no influence of extension and contraction in a region other than the measurement region.

As for the strain sensor 100i, the configuration and features other than the above can be similar to the strain sensor 100a of the first embodiment.

### Tenth Embodiment

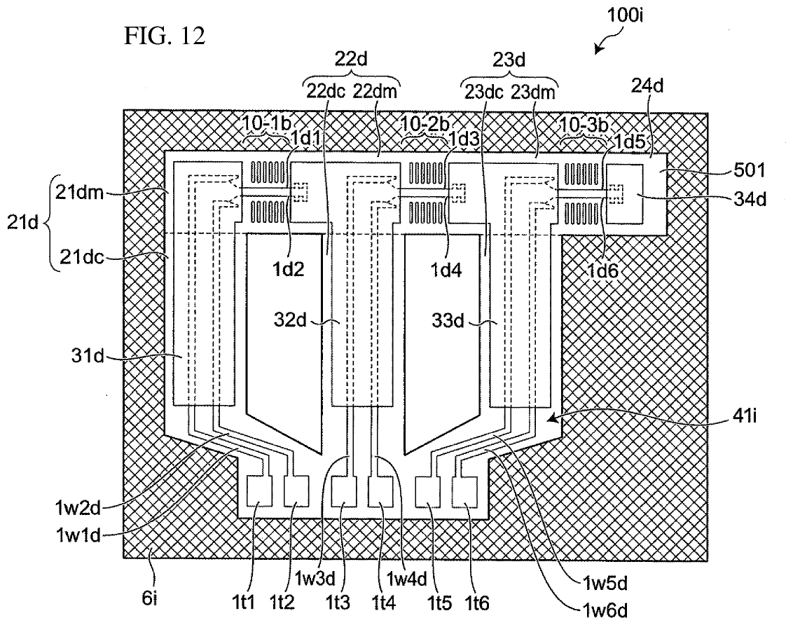

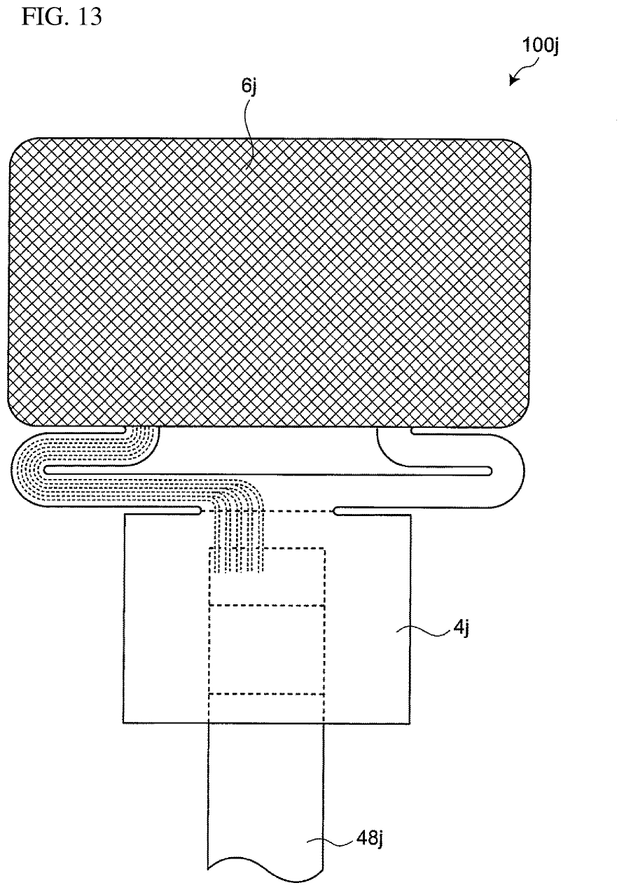

As shown in FIG. 13, a strain sensor 100j of a tenth embodiment includes a sensor unit 4j and a fixing member 6j. In plan view, the outer shape of the fixing member 6j overlaps the outer shape of the sensor sheet 41j. FIG. 13 is a plan view of the strain sensor 100j when viewed from the back side, that is, a plan view of the strain sensor 100j when viewed from the fixing member 6j side.

As for the strain sensor 100j, the configuration and features other than the fixing member 6j can be similar to the strain sensor 100a of the first embodiment.

In the strain sensor 100j configured as described above, the outer shape of the fixing member 6j is the same as the outer shape of the sensor sheet 41j, so handling is easy.

### Eleventh Embodiment

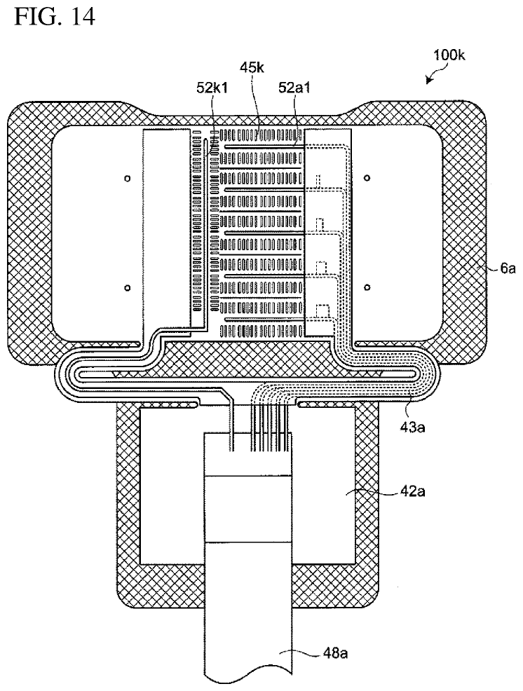

As shown in FIG. 14, a strain sensor 100k of an eleventh embodiment includes not only the detection conductor 52a1 but also another detection conductor 52k1 in the sensing portion of the strain sensor 100a of the first embodiment. The detection conductor 52a1 and the other detection conductor 52k1 expand and contract in different directions.

Specifically, the strain sensor of the eleventh embodiment includes a plurality of detection portions, specifically, six detection portions, in the sensing portion of the sensor sheet 41k. Five detection portions among the plurality of detection portions are disposed parallel to one another, and the remaining one detection portion is disposed so as to intersect substantially perpendicularly with regions extended from all the parallel detection portions in a length direction. More specifically, near the distal ends of the parallel detection conductors 52a1, the other detection conductor 52k1 is disposed substantially parallel to the overall line connecting the distal ends.

The other detection conductor 52k1 may have a function to detect the orientation of an object to be measured, different from the detection conductors 52a1 that detect a strain of the object to be measured. When, for example, the strain sensor of the present embodiment is used as a swallowing sensor, not only the detection of the motion of a throat of a subject with a detection conductor but also the detection of the up and down motion of a jaw with another detection conductor (hereinafter, also referred to as “posture detection conductor”) can be performed, and an influence due to the motion can be corrected, so a strain of an object to be measured is highly accurately detected.

In other words, the present disclosure provides a strain sensor provided with a sensing portion including a plurality of detection portions. At least one of the detection portions and the other of the detection portions expand and contract in different directions.

In a predetermined mode, at least some of the plurality of detection portions are disposed parallel to each other, and another one or some of the detection portions is disposed so as to intersect with the regions extended from all the parallel detection portions in a length direction.

In the present embodiment, as shown in FIG. 14, the number of the posture detection conductors is one; however, the configuration is not limited thereto, and the number of the posture detection conductors may be multiple, for example, two, three or four.

In the present embodiment, the posture detection conductor is disposed perpendicularly to the detection conductor; however, the configuration is not limited thereto, and both just need to expand and contract in different directions and be capable of detecting strains in different directions. For example, an angle made between the detection conductor and an expansion and contraction direction of the posture detection conductor may be greater than or equal to 10°, preferably greater than or equal to 45°, more preferably greater than or equal to 70°, further preferably greater than or equal to 80°, and particularly preferably 90°.

### Twelfth Embodiment

A strain sensor of a twelfth embodiment is a strain sensor including a sensor sheet provided with a sensing portion including a detection portion that expands and contracts in a predetermined direction according to a strain of an object to be measured and that detects a strain in the expansion and contraction direction, and a non-sensing portion that is located on each end of the sensing portion and that supports the sensing portion. The sensing portion is easier to deform than the non-sensing portion.

In one mode, where a Young's modulus of the sensing portion is Y1, a thickness of the sensing portion is T1, a Young's modulus of the non-sensing portion is Y2, and a thickness of the non-sensing portion is T2, a product F1 of Y1 and T1 is less than a product F2 of Y2 and T2. Here, the Young's modulus means an apparent Young's modulus.

In a preferred mode, the ratio of F1 to F2 (F1/F2) can be lower than or equal to 0.06 and preferably lower than or equal to 0.03.

In a preferred mode, the strain sensor of the twelfth embodiment may further include a fixing member having a first main surface and a second main surface opposite to the first main surface.

In the above mode, the sensor sheet is fixed so as to at least partially overlap the first main surface of the fixing member, and, in plan view, a portion at which the sensing portion and the fixing member overlap is easier to deform than a portion at which the non-sensing portion and the fixing member overlap.

In the strain sensor of the twelfth embodiment, by making the sensing portion easier to deform than the non-sensing portion, for example, by setting a product of the Young's modulus and thickness of the sensing portion to a value less than a product of the Young's modulus and thickness of the non-sensing portion, detection of a strain that occurs in low-elastic physical characteristics, for example, a strain caused by a bulge of a skin or the like, detection of the motion of a throat when swallowing, particularly, detection of forward movement of laryngeal prominence can be further accurately performed. As in the case of the first embodiment, by forming slits in the sensing portion, the sensing portion is easier to deform than the non-sensing portion. As another method, the sensing portion may be made easier to deform than the non-sensing portion by reducing the thickness of the substrate in the sensing portion as compared to the non-sensing portion or reducing the width of the sensing portion as compared to the non-sensing portion. In the strain sensor according to the present disclosure, the sensing portion may be made easy to deform by providing a plurality of through-holes or forming a recessed portion in a groove shape or in a dot-like shape, instead of providing slits. Even when a fixing member is present, a portion at which the sensing portion and the fixing member overlap in plan view is made easier to deform than a portion at which the non-sensing portion and the fixing member overlap, for example, a product of the Young's modulus and thickness of the sensing portion is made less than a product of the Young's modulus and thickness of the non-sensing portion. Thus, the detection of a strain that occurs in low-elastic physical characteristics as in the case of the above is possible.

In the strain sensor according to the present disclosure, the sensing portion having a higher time response is preferably used since the detection accuracy improves. A “time response” is an index indicating a time difference of an output to an input, and a time response is better as a time difference decreases. In the strain sensor according to the present disclosure, a strain deformation is an input, and a detection signal is an output. A process until the output is such that the sensing portion deforms following a strain deformation of an object to be measured to output a detection signal according to the deformation. Therefore, accurately, a time response is determined depending on a deformation of the sensing portion for a strain deformation of an object to be measured, and a time difference between detection signals for a deformation of the sensing portion. Here, even when a sensing portion having a good time response is adopted, a time difference may occur in a change of the shape of the fixing member for an input deformation, and, particularly in the case of a contraction strain deformation, it appears as a slack of the fixing member. When such a fixing member is used, the time response of the sensing portion is decreased. In detecting a successive expansion and contraction strain deformation in the strain sensor according to the present disclosure, when an expansion deformation is inputted in a state where a slack during the last contraction deformation is remaining, a deformation of the sensing portion does not follow a deformation of an object to be measured until the slack is removed, so a detection signal cannot be outputted. For this reason, when a fixing member having smaller hysteresis of elastic modulus during expansion and contraction than the hysteresis of elastic modulus during expansion and contraction of the sensing portion is used, the time response of the sensing portion is not decreased, so it is preferable.

In the strain sensors of the first to twelfth embodiments, by decreasing the elastic modulus of the whole of a measurement portion with the low-elastic modulus portion including slits, detection of a strain that occurs in low-elastic physical characteristics, for example, a strain caused by a bulge of a skin or the like is made possible. However, the present disclosure is not limited thereto. Instead of forming a low-elastic modulus portion, for example, the elastic modulus of the measurement portion may be decreased by reducing the thickness of the substrate in the measurement portion as compared to the non-sensing portion or reducing the width of the measurement portion as compared to the non-sensing portion. In the strain sensor according to the present disclosure, in the low-elastic modulus portion, the elastic modulus of the measurement portion may be decreased by providing a plurality of through-holes or forming a recessed portion in a groove shape or dot-like shape instead of slits. In the specification, a low-elastic modulus portion includes reducing the elastic modulus of the whole of the measurement portion by reducing the thickness of the substrate in the measurement portion as compared to the non-sensing portion or reducing the width of the measurement portion as compared to the non-sensing portion.

In the strain sensors of the first to twelfth embodiments, the detection portion is made up of a detection conductor and is a so-called electric sensor. However, the detection portion of the strain sensor according to the present disclosure is not limited, and, for example, an optical sensor may be used.

In a preferred mode, the strain sensor according to the present disclosure has a sticking member on the second main surface of the non-sensing portion.

The sticking member is preferably an adhesive layer made of an adhesive material.

The adhesive material is not limited and may be, for example, an acrylic or silicone sticking material having high elasticity. In a preferred mode, the adhesive material is a biocompatible adhesive material with no cytotoxicity, for example, 1524 made by 3M Company.

In the specification, an apparent Young's modulus and hysteresis are measured as follows. A strip sample of which the cross-sectional shape has a thickness of t and a width of W is prepared. The strip sample is expanded to a strain of ε at a tension rate of 1 mm/s, and then a tensile load F at the time when the strip sample is contracted to an initial length is measured. From the result of measurement, stress (Pa), apparent Young's modulus, the hardness of each member, and hysteresis can be determined as follows. Stress (Pa): Tensile load F (kgf)×Gravitational acceleration 9.8 (mm/s²)×Thickness (mm)×Width W (mm) Apparent Young's modulus: σ/ε when stress at a maximum strain of ε is σ Hardness of each member: Apparent Young's modulus×Thickness t Hysteresis: When stress at a maximum strain of ε is σ, stress at a strain of ε/2 during tension is σ1, and stress at a strain of ε/2 during contraction is σ2, the ratio of a difference between σ1 and σ2 to σ, that is, (σ1−σ2)/σ

The strain sensor according to the present disclosure is usable in detecting the motion of a throat when swallowing.

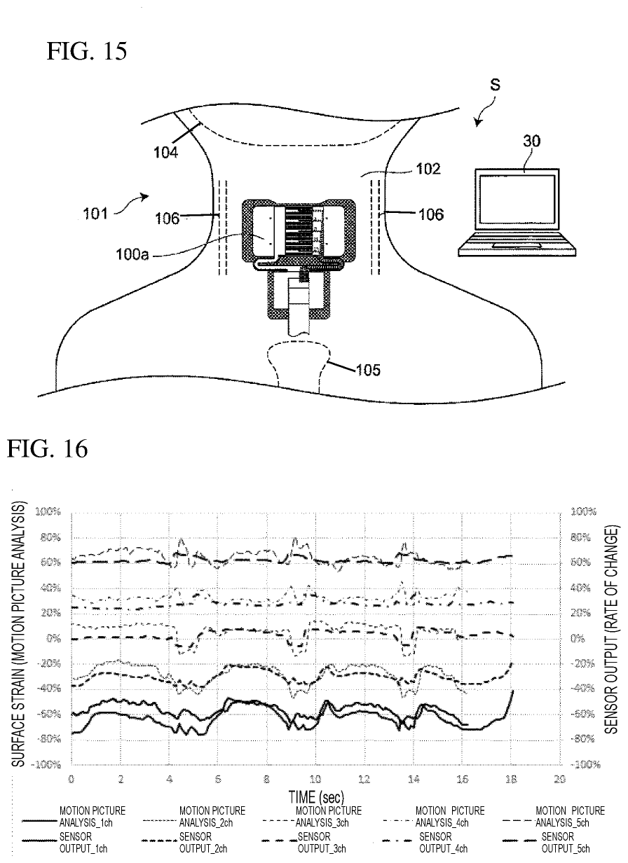

As shown in FIG. 15, the sensing portion of the strain sensor is attached to the skin of an anterior neck 102 of a subject 101 so as to cover the range of the motion of thyroid cartilage, which occurs with swallowing. A lower jawbone 104 is located above the thyroid cartilage, and a breast bone 105 is located below. A pair of carotid arteries 106 is located on both right and left sides of the thyroid cartilage. The sensing portion is disposed in a range that does not overlap the lower jawbone 104, the breast bone 105, and the carotid arteries 106. The sensing portion deforms due to a displacement of the thyroid cartilage caused by swallowing of the subject 101. For example, in one swallowing motion, the thyroid cartilage rises upward about 20 mm from the position before the swallowing motion, moves forward, and then lowers and returns to the original position.

In the above usage, the strain sensor determines swallowing by determining upward movement and forward movement of the laryngeal prominence in accordance with a signal obtained from the detection portion provided in the sensing portion. The sensing portion is made up of a plurality of detection conductors, and the expansion and contraction direction to be detected is disposed in a direction perpendicular to the up and down movement direction of the thyroid cartilage. When the thyroid cartilage is adjacent to any one of the detection conductors, the detection conductor is expanded by the amount of protrusion caused by the shape of the thyroid cartilage, with the result that the resistance value of the detection conductor increases. The resistance value is maximum when the largest protruding portion of the thyroid cartilage is located just under the detection conductor. Therefore, when the thyroid cartilage moves up and down so as to cross the detection conductor, a time change in the resistance value of the detection conductor exhibits a peak behavior with a local maximum at a time at which the thyroid cartilage is just under the detection conductor, so a time at which the thyroid cartilage has passed just below the detection conductor can be estimated by back calculation from the resistance value local maximum. In addition, when a plurality of detection conductors is arranged parallel at predetermined intervals and the thyroid cartilage successively passes by the detection conductors with one up and down motion, the moving direction and moving speed of the thyroid cartilage can be estimated from a time difference between the resistance local maximums of the detection conductors. When the thyroid cartilage moves forward and backward, the detection conductor is expanded by a large amount according to the movement, so the resistance value increases. Therefore, the amount of forward and backward movement of the thyroid cartilage can be estimated from the magnitude of the resistance value of the detection conductor. The strain sensor according to the present disclosure is capable of further accurately capturing a deformation in a direction perpendicular to the main surface of the strain sensor, so not only the upward movement of the laryngeal prominence but also the forward movement can be detected, so further accurate swallowing determination can be made.

The swallowing sensor may include a main body portion. The main body portion may be provided on the lower side of the strain sensor. The main body portion is operated by a built-in battery. As the detection portion of the strain sensor obtains a signal, the main body portion performs determination as to whether swallowing is detected in accordance with the signal detected from the detection portion of the strain sensor. When the main body portion detects swallowing, the main body portion extracts data of a signal at the time of the swallowing detection and wirelessly outputs the data to an external device. Determination as to whether swallowing is detected is to determine the presence or absence of swallowing.

The main body portion includes a preprocessing section, a signal processing section, a wireless communication module, a battery, and the like. In this case, the main body portion is detachably connected to the strain sensor by using a connector (not shown) or the like. Thus, when the strain sensor alone is broken or gets dirty, only the strain sensor can be detached from the main body portion for replacement. The placement of the main body portion is not limited to the lower side of the strain sensor. The main body portion may be placed on the right side or the left side of the strain sensor.

The preprocessing section converts the resistance value of each detection conductor of the strain sensor to a signal. The preprocessing section executes a process of supplying a constant voltage or a constant current to each detection conductor and converting an analog output voltage of the detection conductor to a digital signal through AD conversion.

The signal processing section determines the motion of swallowing. For example, data at the time of swallowing may be set as, for example, a data range in which a change in the signal strength of a displacement rate component exceeds a threshold. Alternatively, data at the time of swallowing may be set as, for example, a data range corresponding to a change pattern that matches a preset reference pattern of swallowing (a data range from a swallowing start point of the reference pattern to a swallowing end point). Alternatively, data at the time of swallowing may be set as a data range in which data in a predetermined time before and after any one of the above-described two data ranges is added to the any one of the two data ranges.

The extracted signal is wirelessly outputted by using the wireless communication module. In addition to this, the extracted signal is saved in a memory (storage section) provided inside the main body portion. The wireless communication module is provided in the main body portion and connected to the signal processing section. The wireless communication module includes a modulation circuit that modulates a signal in accordance with various wireless communication standards, a transmission section that transmits a modulated signal (both of which are not shown), and the like. The wireless communication module outputs a signal at the time of swallowing, extracted by the signal processing section, toward a swallowing analyzer 30 as an external device. The swallowing analyzer 30 performs swallowing function analysis by using the received data. A swallowing function analysis is to determine the ability of swallowing, for example, how strong the swallowing force, or the like is.

In the present mode, as the detection portion of the strain sensor obtains a signal, the main body portion performs determination as to whether swallowing is detected in accordance with the signal detected from the detection portion of the strain sensor. Each time the main body portion determines that swallowing is detected, the main body portion extracts data of a signal at the time of the swallowing and wirelessly outputs the data to an external device. Therefore, data to be wirelessly transmitted is only data at the time of swallowing, so it is not necessary to continuously transmit a large amount of data. Hence, for example, the electric power consumption of the communication module is reduced, so a small low-profile, low-capacity built-in battery can be used.

### EXAMPLES

### Example 1

### Manufacturing of Sensor Unit 4a

Initially, a substrate made of thermoplastic polyurethane including the substrate 51a of a sensor sheet portion, the substrate 57a of a terminal portion, and the substrate 58a of a connection portion was prepared. In the substrate, the substrate 51a of the sensor sheet portion is rectangular with a width of 50 mm, a length of 80 mm, and a thickness of 40 μm. Conductors were formed on one of the main surfaces (first main surface) of the substrate, as shown in FIG. 1 and FIG. 2. Here, in the strain sensor of this example, a 30-mm portion from each end of the substrate 51a portion was set as a non-sensing portion, and a 20-mm portion between the non-sensing portions was set as a sensing portion. The detection conductors 52a1 were formed 10 mm from the right end of the sensing portion. The width of each conductor was set to 1.5 mm, and the interval between two of the detection conductors 52a1 was set to 0.6 mm. The interval between the detection portions was set to 8 mm. The conductors were formed by applying a silver paste containing silver particles and thermosetting resin and then hardening the resin by heating. The conductors were also formed on the connection portion and the terminal portion of the substrate.

Slits were formed with a length of 3 mm and a width of 0.2 mm by CO₂ laser beam machining at a pitch of 0.5 mm in each of the low-elastic modulus portions. In addition, the restraint portion 54a was formed so as to cover the wiring conductors in the non-sensing portion 46a, and the restraint portion 55a was also formed on the non-sensing portion 47a. The restraint portions 54a, 55a each were formed from an UV-curing urethane modified acrylic resin.

The sensor unit 4a obtained as described above was manufactured. A tensile load along the expansion and contraction direction in the sensing portion of the sensor unit was measured. The results are shown in Table 1.

Manufacturing of Strain Sensor Next, the fixing member 6a was prepared. Chloroprene rubber sponge (closed-cell foam) with a thickness of 2 mm was used as the fixing member 6a. A tensile load and a compressive load of the fixing member were measured. The results are shown in Table.

The sensor unit 4a was fixed to the fixing member 6a by attaching the sensor sheet 41a and the terminal portion 42a of the sensor unit 4a obtained as described above to the fixing member 6a with adhesive. Thus, the strain sensor of Example 1 was manufactured. At this time, no tensile stress was applied to the sensor sheet 41a. An acrylic adhesive was used as the adhesive. A tensile load in the sensing portion of the strain sensor of Example 1 was measured. The results are shown in Table 1.

TABLE 1

| | TENSILE LOAD (N/mm) | | | COMPRESSIVE LOAD (N/mm) | | |
|---|---|---|---|---|---|---|
| (STRAIN) | 5% | 10% | 20% | 5% | 10% | 20% |
| SENSING PORTION | 0.016 | 0.026 | 0.036 | — | — | — |
| FIXING MEMBER | 0.042 | 0.084 | 0.16 | 0.01 | 0.02 | 0.04 |
| STRAIN SENSOR | 0.065 | 0.11 | 0.19 | — | — | — |
| (SENSING PORTION) | | | | | | |

### Test Example 1

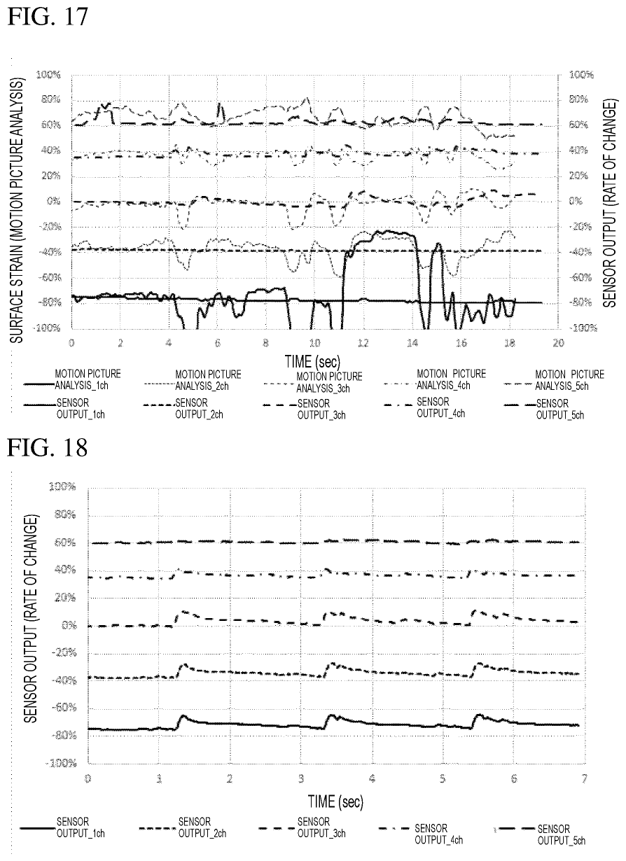

Two different persons each having a different hardness of a skin of a throat were a subject A and a subject B, the sensor sheet 41a with no fixing member was directly attached to the throat, and the motion of the throat at the time of swallowing water was measured. The actual motion of the skin surface was analyzed by motion picture analysis. FIG. 16 shows the results of the subject A. FIG. 17 shows the results of the subject B.

It was found from the results that, for the subject A, the output change similar to the motion picture analysis results was observed and, for the subject B, some of the sensors did not output a change and could not detect a strain. When the states where the sensor sheet was attached were compared, the subject A having a hard skin was in a state where both the throat and the sensor sheet had no wrinkles, whereas the subject B having a soft skin was in a state where the throat had wrinkles and the sensor sheet was contracted. The surface of the throat deforms depending on the motion of the internal cartilage. However, since the subject B has a soft skin, it is presumable that the sensor sheet portion was constantly contracted and not deformed and, therefore, a deformation of the throat was not sufficiently transferred to the sensor sheet and a measurement malfunction was occurring.

For this reason, the strain sensor 100a of Example 1 with the fixing member was attached (1) in no wrinkle state and (2) in a state where wrinkles were intentionally formed, to the throat of the subject B, and the motion of the throat at the time of swallowing water was measured. FIG. 18 shows the results in the case of (1) no wrinkle state. FIG. 19 shows the results in the case where (2) wrinkles were intentionally formed.

It was demonstrated from the above results that similar results were obtained from any of the cases (1) and (2) and the motion was stably detected with the strain sensor of the present application regardless of the state of the throat.

### Example 2

A strain sensor of Example 2 was manufactured as in the case of Example 1 except that the sensor unit 4a was fixed to the fixing member 6a in a state where a tensile stress (0.036 N/mm at a strain of 20%) was applied to the sensor sheet 41a.

### Test Example 2

For each of the strain sensor of Example 1 and the strain sensor of Example 2, a strain was applied between 0% and 20% at a repetition cycle of about three times in 50 seconds, and a change in resistance value to the strain was measured.

It was demonstrated from the above results that, when the strain sensor of Example 2 was used, the resistance value at a strain of 0% was not changed after a strain was repeatedly applied. On the other hand, it was demonstrated that, in Example 1, the resistance value at a strain of 0% was increasing as a strain was repeatedly applied. In other words, it was demonstrated that no zero drift was occurring in Example 2.

### Example 3, Example 4, and Comparative Example 1

Strain sensors (Example 3, Example 4, and Comparative Example 1) were manufactured by using a sensor unit having a similar configuration to the sensor unit of Example 1 and changing the thickness of the sensor sheet, the shape of each slit, and the material of the fixing member to adjust the Young's modulus of the sensing portion, the Young's modulus of each non-sensing portion, the hysteresis of the elastic modulus at the time of expansion and contraction of the fixing member, and the hysteresis of elastic modulus at the time of expansion and contraction of the sensor sheet to the values in the following Table.

TABLE 2

| | | SENSING | NON-SENSING | FIXING |
|---|---|---|---|---|
| | | PORTION | PORTION | MEMBER |
| EXAMPLE 3 | MEMBER | THERMOPLASTIC | THERMOPLASTIC | ETHYLENE |
| | USED | POLYURETHANE | POLYURETHANE | PROPYLENE |
| | | (WITH SLITS) | (RESTRAINT | RUBBER |
| | | | LAYER) | SPONGE |
| | YOUNG'S | 15.7 MPa | 198.4 MPa | 1.0 MPa |
| | MODULUS | | | |
| | YOUNG'S | 0.6N/mm | 29.8N/mm | 3.0N/mm |
| | MODULUS × | | | |
| | THICKNESS | | | |
| | HYSTERESIS | 25.0% | 23.7% | 12.4% |
| EXAMPLE 4 | MEMBER | THERMOPLASTIC | THERMOPLASTIC | ETHYLENE |
| | USED | POLYURETHANE | POLYURETHANE | PROPYLENE |
| | | (LAMINATE OF | (RESTRAINT | RUBBER |
| | | THREE LAYERS) | LAYER) | SPONGE |
| | YOUNG'S | 37.7 MPa | 198.4 MPa | 1.0 MPa |
| | MODULUS | | | |
| | YOUNG'S | 4.5N/mm | 29.8N/mm | 3.0N/mm |
| | MODULUS × | | | |
| | THICKNESS | | | |
| | HYSTERESIS | 17.5% | 23.7% | 12.4% |
| COMPARATIVE | MEMBER | THERMOPLASTIC | THERMOPLASTIC | ETHYLENE |
| EXAMPLE 1 | USED | POLYURETHANE | POLYURETHANE | PROPYLENE |
| | | (LAMINATE OF | (SINGLE LAYER) | RUBBER |
| | | THREE LAYERS) | | SPONGE |
| | YOUNG'S | 37.7 MPa | 37.7 MPa | 1.0 MPa |
| | MODULUS | | | |
| | YOUNG'S | 4.5N/mm | 1.5N/mm | 3.0N/mm |
| | MODULUS × | | | |
| | THICKNESS | | | |
| | HYSTERESIS | 17.5% | 17.5% | 12.4% |

As shown in the Table, in each of the strain sensor, the product of Young's modulus and thickness and the hysteresis have the following relationship. In Example 3, the product of the Young's modulus and the thickness of the sensing portion is less than the product of the Young's modulus and the thickness of each non-sensing portion, and the hysteresis of elastic modulus at the time of expansion and contraction of the fixing member is smaller than the hysteresis of elastic modulus at the time of expansion and contraction of the sensor sheet. In Example 4, the product of the Young's modulus and the thickness of the sensing portion is less than the product of the Young's modulus and the thickness of each non-sensing portion, and the hysteresis of elastic modulus at the time of expansion and contraction of the fixing member is smaller than the hysteresis of elastic modulus at the time of expansion and contraction of the sensor sheet. In Comparative Example 1, the product of the Young's modulus and the thickness of the sensing portion is greater than the product of the Young's modulus and the thickness of each non-sensing portion, and the hysteresis of elastic modulus at the time of expansion and contraction of the fixing member is smaller than the hysteresis of elastic modulus at the time of expansion and contraction of the sensor sheet.

### Test Example 3

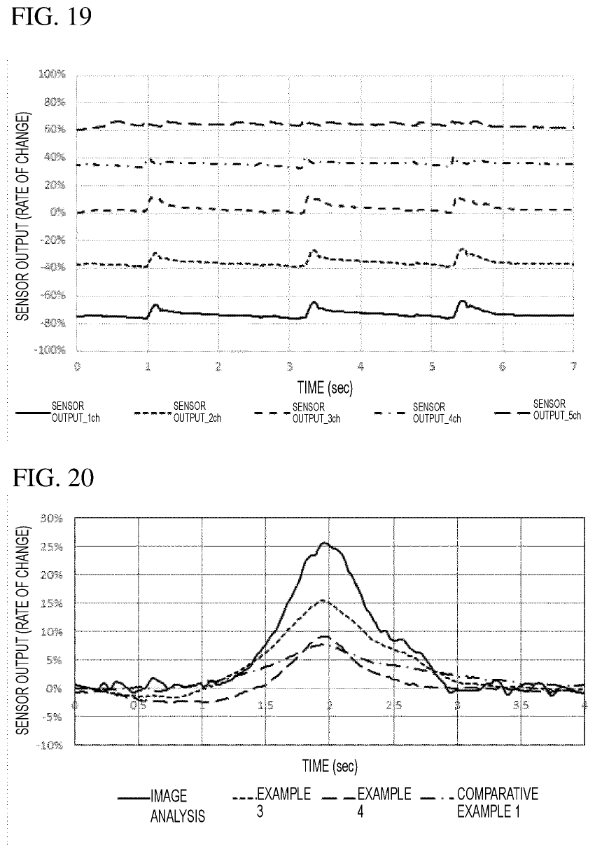

The strain sensor was attached to the throat of a subject, and the motion of the throat at the time of swallowing water was measured. FIG. 20 shows the results. In FIG. 20, the dashed line represents the results obtained by measuring a strain of the surface of the skin through video analysis when the thyroid cartilage moves forward and backward at the time of swallowing.

As shown in FIG. 20, Comparative Example 1 output from time before a change in strain and provided broad characteristics in which the output did not coincide with a baseline even after the change of a strain, and the strain detection accuracy was low. In contrast, Example 3 and Example 4 had large outputs for a strain, detected a peak, and the strain detection accuracy was high.

The ratio (F1/F2) of the product (F1) of the Young's modulus and thickness of the sensing portion to the product (F2) of the Young's modulus and thickness of each non-sensing portion of Example 3 was 0.02, F1/F2 of Example 4 was 0.15, and Example 3 having a lower value of F1/F2 had a higher peak.

The strain sensor according to the present disclosure is applicable to usages desired to detect a strain in various regions, for example, detecting a deformation of a local bulge or the like of a skin of a human body.
100a to 100k strain sensor
1 conductor portion
1cd3 third conductor
1cd4 fourth conductor
1cd5 fifth conductor
1cd6 sixth conductor
1t connection terminal conductor
1w wiring conductor
1d, 1da detection conductor
1t1 to 1t6 first to sixth connection terminal conductors
1w1 to 1w6, 1w1d to 1w6d first to sixth wiring conductors
1d1 to 1d6 first to sixth detection conductors
3, 3-1 to 3-10 slit
4a, 4j sensor unit
6a to 6j fixing member
10, 10a sensing portion
10-1, 10-1a, 10-1b first sensing portion
10-2, 10-2a, 10-2b second sensing portion
10-3, 10-3a, 10-3b third sensing portion
11, 11-1 to 11-6, 11a detection portion
12, 12-1 to 12-6, 12a low-elastic modulus portion
12a1 first low-elastic modulus portion
12a2 second low-elastic modulus portion
20 non-sensing portion
21a, 21b, 21c, 21d first non-sensing portion
21b0 base non-sensing portion
21b1 first branch non-sensing portion
21b2 second branch non-sensing portion
21dc first wiring non-sensing portion
21dm first measurement non-sensing portion
22a, 22b, 22c, 22d second non-sensing portion
22dc second wiring non-sensing portion
22dm second measurement non-sensing portion
23b, 23c, 23d third non-sensing portion
23dc third wiring non-sensing portion
23dm third measurement non-sensing portion
24b, 24c, 24d fourth non-sensing portion
31a, 31c, 31d first restraint portion
32a, 32c, 32d second restraint portion
33c, 33d third restraint portion
34b restraint portion
34c, 34d fourth restraint portion
41a, 41c to 41j sensor sheet
42a terminal portion
43a connection portion
45a, 45k sensing portion
46a, 47a non-sensing portion
48a, 48j flat cable
51a substrate
52a conductor
52a1, 52k1 detection conductor
52a2 fixed conductor
52a3 wiring conductor
52a4 terminal conductor
53a slit
54a, 55a restraint portion
57a substrate
58a substrate
61b window
101, 201, 301, 401, 501 substrate

1. A strain sensor comprising:
a sensor sheet provided with a sensing portion including a detection portion, wherein the detection portion expands and contracts in a predetermined direction according to a strain of an object to be measured and detects a strain in the expansion and contraction direction; and
a fixing member having a first main surface and a second main surface opposite to the first main surface, wherein
the sensor sheet is fixed so as to at least partially overlap the first main surface of the fixing member, and
a tensile load of the fixing member is greater than a tensile load of the sensing portion of the sensor sheet.

2. The strain sensor according to claim 1, wherein the tensile load of the sensing portion is less than a tensile load of the object to be measured.

3. The strain sensor according to claim 1, wherein
a tensile load of the strain sensor in a region in which the sensing portion is present is less than or equal to 0.10 N/mm at a strain of 5%, less than or equal to 0.15 N/mm at a strain of 10%, and less than or equal to 0.25 N/mm at a strain of 20% along an expansion and contraction direction of the detection portion, and
a compressive load of the fixing member is greater than or equal to 0.005 N/mm at a strain of 5%, greater than or equal to 0.01 N/mm at a strain of 10%, and greater than or equal to 0.03 N/mm at a strain of 20% along the expansion and contraction direction of the detection portion.

4. A strain sensor comprising:
a sensor sheet provided with a sensing portion and a non-sensing portion, wherein the sensing portion includes a detection portion, the detection portion expands and contracts in a predetermined direction according to a strain of an object to be measured and detects a strain in the expansion and contraction direction, and the non-sensing portion is located on each end of the sensing portion and supports the sensing portion, wherein
the sensing portion is easier to deform than the non-sensing portion.

5. The strain sensor according to claim 4, wherein, where a Young's modulus of the sensing portion is Y1, a thickness of the sensing portion is T1, a Young's modulus of the non-sensing portion is Y2, and a thickness of the non-sensing portion is T2, a product F1 of Y1 and T1 is less than a product F2 of Y2 and T2.

6. The strain sensor according to claim 4, further comprising:
a fixing member having a first main surface and a second main surface opposite to the first main surface, wherein
the sensor sheet is fixed so as to at least partially overlap the first main surface of the fixing member, and in plan view, a portion at which the sensing portion and the fixing member overlap is easier to deform than a portion at which the non-sensing portion and the fixing member overlap.

7. The strain sensor according to claim 1, wherein the fixing member is a sponge material.

8. The strain sensor according to claim 7, wherein a thickness of the fixing member is greater than or equal to 1 mm and less than or equal to 5 mm.

9. The strain sensor according to claim 1, wherein an outer shape of the fixing member and an outer shape of the sensor sheet overlap in plan view.

10. The strain sensor according to claim 1, wherein the fixing member is present so as to at least overlap an entire portion of the sensor sheet in plan view.

11. The strain sensor according to claim 1, wherein the fixing member is present so as to surround the sensing portion of the sensor sheet in plan view.

12. The strain sensor according to claim 1, wherein the detection portion comprises a plurality of detection portions.

13. The strain sensor according to claim 12, wherein the plurality of detection portions is disposed parallel to each other.

14. The strain sensor according to claim 1, wherein the detection portion comprises a plurality of detection portions, the sensing portion includes the plurality of the detection portions, and at least one of the detection portions and another one of the detection portions expand and contract in different directions.

15. The strain sensor according to claim 14, wherein at least some of the plurality of detection portions are disposed parallel to each other, and others of the detection portions are disposed so as to intersect with a region extending in a length direction from all of the detection portions disposed parallel to each other.

16. The strain sensor according to claim 12, wherein the plurality of detection portions is disposed such that expansion and contraction directions of the detection portions are radial.

17. The strain sensor according to claim 1, wherein the detection portion is a detection conductor having a resistance value changeable according to expansion and contraction of the detection portion.

18. The strain sensor according to claim 1, wherein the sensing portion is placed in a state where a tensile stress is applied along an expansion and contraction direction of the detection portion.

19. The strain sensor according to claim 1, wherein the sensing portion has a plurality of slits provided in a direction intersecting with an expansion and contraction direction of the detection portion.

20. The strain sensor according to claim 1, wherein a hysteresis of an elastic modulus of the fixing member during expansion and contraction of the fixing member is smaller than a hysteresis of an elastic modulus of the sensing portion during expansion and contraction of the sensing portion.
